(19) United States
(12) Patent Application Publication
(10) Pub. No.: US 2026/0335635 A1
(43) Pub. Date: Oct. 29, 2026

(54) TISSUE REMOVAL DEVICE FOR HELIX LEADS

(71) Applicant: Medtronic, Inc., Minneapolis, MN (US)

(72) Inventors: Kevin L. Sack, New Brighton, MN (US); Andrew J. Ries, Lino Lakes, MN (US); Tina M. Billstrom, Ham Lake, MN (US); Ryan E. Begley, Minneapolis, MN (US); Emily G. Klotsche, St. Paul, MN (US)

(21) Appl. No.: 19/652,844

(22) Filed: Apr. 20, 2026

Related U.S. Application Data
(60) Provisional application No. 63/794,577, filed on Apr. 25, 2025.

(51) Int. Cl. A61N 1/08; A61N 1/04

(52) U.S. Cl. CPC A61N 1/08; A61N 1/0488

(57) ABSTRACT

A device includes a frame defining an engagement zone configured to receive a distal portion of a medical lead having a helix at a distal end. An engagement element couples to the frame. An actuation assembly transitions the engagement element from a first configuration to a second configuration, where the engagement element contacts the helix in the second configuration. Relative movement of the helix and the engagement element removes patient tissue from the helix.

This application claims the benefit of U.S. Provisional Patent Application No. 63/794,577, filed Apr. 25, 2025, the entire content of which is incorporated herein by reference.

### TECHNICAL FIELD

This disclosure relates generally to techniques for implanting a medical device in a patient and, more particularly to systems, devices, and methods for removing tissue from a fixation element of a medical lead, e.g., during a procedure for implanting the lead.

### BACKGROUND

Implantable medical devices (IMDs), such as pacemakers, implantable cardioverter-defibrillators (ICDs), and cardiac resynchronization therapy (CRT) devices, may provide therapy for treating various cardiac conditions. IMD systems may include a can and one or more medical leads. The one or more medical leads may extend from the can of the IMD to a target location within a heart of a patient. The one or more medical leads may sense electrical activity and/or deliver therapeutic electrical stimulation. Each medical lead may have a fixation element, such as a helix, which may secure a distal end of the medical lead to the heart of the patient. For example, during an implantation procedure, a physician navigates the medical lead to the target location and anchors the medical lead to the heart via the fixation element. The fixation element may serve as an electrode for sensing electrical activity or delivering therapeutic electrical stimulation. The target location may include the His bundle, the left bundle branch, or other part of the conduction system of the heart.

### SUMMARY

This disclosure describes devices, systems, methods, and techniques related to removing patient tissue from a fixation element of a medical lead. The fixation element may include a fixation helix. A cleaning device may include a frame defining an engagement zone configured to receive a distal portion of the medical lead. An engagement element, which may include one or more strands, a plurality of bristles, a foam material, or any other element which is suitable to engage the patient tissue, may couple to the frame within or adjacent to an engagement zone. An actuation assembly may transition the engagement element from a first configuration, such as an open or loading configuration, to a second configuration, such as a cleaning or engagement configuration. In the second configuration, the engagement element may contact the helix and immobilize the patient tissue located on the helix.

Operation of the cleaning device may involve a user positioning the helix within the engagement zone and the user actuating the cleaning device to engage the engagement element with the patient tissue on the helix. The user may rotate the medical lead relative to the engaged cleaning device which may cause the helix to withdraw from the engagement element. This relative movement may separate the patient tissue from the helix while maintaining an integrity of the medical lead. The cleaning device may utilize a threaded interface to define a rate of withdrawal to match a pitch of the helix, or the device may enable manual rotation and withdrawal by a user.

The techniques of the disclosure facilitate efficient cleaning of the helix while protecting an integrity of the helix and/or protecting sensitive components (e.g., a drug-eluting collar) of the medical lead. For example, manual cleaning attempts using non-specialized tools may require up to ten minutes per implantation attempt, and a given implantation procedure may require multiple attempts. Furthermore, manual techniques may utilize tools not designed for the task, which increases the risk of damaging lead components, such as the Monolithic Controlled Release Device (MCRD) rings. The frame may define an alignment window configured to provide visual confirmation of a lead insertion depth. Aligning a feature of the medical lead, such as a ring electrode, with the alignment window may facilitate positioning of the helix for cleaning. The actuation assembly may include a mechanical stop to limit a tension or pressure applied to the helix by the engagement element. The actuation assembly may additionally include a spring to limit a tension applied to the helix by the engagement element.

In one example, this disclosure is directed to a device comprising a frame defining an engagement zone configured to receive a distal portion of a medical lead, the medical lead comprising a helix at a distal end of the medical lead, an engagement element coupled to the frame, and an actuation assembly configured to transition the engagement element from a first configuration to a second configuration, wherein the engagement element in the second configuration is configured to contact the helix such that relative movement of the helix and the engagement element removes patient tissue from the helix.

In another example, this disclosure describes a method comprising inserting a helix of a medical lead into an engagement zone of a frame of a cleaning device, wherein an engagement element of the cleaning device is in a loading configuration, transitioning the cleaning device from the loading configuration to a cleaning configuration, wherein the engagement element is configured to immobilize patient tissue in the helix in the cleaning configuration, and rotating the medical lead relative to the engagement element while maintaining the cleaning configuration to separate the patient tissue from the helix.

In another example, this disclosure is directed to a system comprising a medical lead comprising a helix at a distal end of the medical lead, and a cleaning device comprising a frame defining an engagement zone configured to receive the distal end of the medical lead, an engagement element coupled to the frame, and an actuation assembly configured to transition the engagement element from a first configuration to a second configuration, wherein the engagement element in the second configuration contacts the helix such that relative movement of the helix and the engagement element removes patient tissue from the helix.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram illustrating an example cardiac system configured to connect to cardiac tissue of a patient.

FIG. 2 is a conceptual drawing illustrating a medical lead of FIG. 1 with a helix, in accordance with one or more techniques of this disclosure.

FIG. 3A is a perspective view illustrating an example cleaning device in a loading configuration without strands, in accordance with one or more techniques of this disclosure.

FIG. 3B is a perspective view illustrating the example cleaning device of FIG. 3A in a cleaning configuration with strands engaging a helix of a medical lead.

FIG. 4A is a front view illustrating an example frame of the example cleaning device of FIG. 3A.

FIG. 4B is a back view illustrating the example frame of the example cleaning device of FIG. 3A.

FIG. 5A is a first side view illustrating a first frame portion of the example cleaning device of FIG. 3A.

FIG. 5B is a second side view illustrating a second frame portion of the example cleaning device of FIG. 3A.

FIG. 6A is a top view illustrating the example cleaning device of FIG. 3A.

FIG. 6B is a cross-sectional view of the example cleaning device frame taken along line 6B-6B of FIG. 6A.

FIG. 7A is a perspective view illustrating an example cleaning device in a loading configuration.

FIG. 7B is a perspective view illustrating the example cleaning device of FIG. 7A with strands in the loading configuration around the helix.

FIG. 8A is a side view illustrating the example cleaning device of FIG. 7A.

FIG. 8B is an exploded side view illustrating the example cleaning device of FIG. 8A.

FIG. 8C is a cross-sectional view of the example cleaning device of FIG. 8A.

FIG. 9A is a rotated side view illustrating the example cleaning device of FIG. 7A.

FIG. 9B is an exploded side view illustrating the example cleaning device of FIG. 9A.

FIG. 9C is a cross-sectional view of the example cleaning device of FIG. 9A.

FIG. 10A is a perspective view illustrating an example handheld cleaning tool.

FIG. 10B is a perspective view illustrating the example handheld cleaning tool of FIG. 10A with a strand wrapped in an engagement pattern around a helix of a medical lead.

FIG. 11A is a side view illustrating the example handheld cleaning tool of FIG. 10A.

FIG. 11B is a cross-sectional view taken along line 11B-11B of FIG. 11A.

FIG. 11C is a cross-sectional view taken along line 11C-11C of FIG. 11A.

FIG. 12A is a front view of the example handheld cleaning tool of FIG. 10A.

FIG. 12B is a top view of the example cleaning tool of FIG. 10A.

FIG. 13A is a conceptual diagram illustrating an example cleaning tool including a brush element in a loading configuration.

FIG. 13B is a conceptual diagram illustrating the example cleaning tool of FIG. 13A in a cleaning configuration.

FIG. 14A is a side view illustrating an example tweezer-style cleaning device in a loading configuration.

FIG. 14B is a side view illustrating the example tweezer-style cleaning device of FIG. 14A in a cleaning configuration.

FIG. 15A is a conceptual diagram illustrating an example cleaning device including a foam material in a loading state.

FIG. 15B is a conceptual diagram illustrating the example cleaning device of FIG. 15A engaging a helix.

FIG. 16 is a flowchart illustrating an example method for removing patient tissue from a helix of a medical lead.

### DETAILED DESCRIPTION

In some cases, if a heart of a patient is not working properly, e.g., a heart arrhythmia, a clinician may choose to implant an implantable medical device (e.g., an IMD). The IMD may be configured to deliver cardiac therapy, such as cardiac resynchronization therapy (CRT), conduction system pacing, bradycardia pacing, or anti-tachycardia pacing or shocks to the patient. Pacing therapy, for example, may include sending electrical pulses to the heart of the patient through a medical lead which may improve the symptoms of the heart arrhythmia. A clinician may secure the medical lead to the heart of the patient via a fixation element, which may include a fixation helix (e.g., a helix).

In some cases, during implantation of medical leads, the clinician may implant and reimplant the medical lead to improve positioning of the helix within the heart, to improve electrical connectivity to the heart, and/or to improve a mechanical strength of the connection between the helix and the heart. As a result of these implantation and re-implantation attempts, the helix of the medical lead may accumulate patient tissue, such as heart tissue. For example, the helix may become clogged with the patient tissue which may result in reduced reimplant success. Removing the heart tissue from the helix before the medical lead is repositioned may improve the success of the reimplantation of the medical lead. In some cases, removing the heart tissue from the helix may be manual and ad hoc. For example, the clinician may utilize non-specialized tools found in the operating room such as hypodermic needles, vein picks, scalpels, gauze, or manual manipulation with gloved fingers. These manual techniques may be time-consuming and inefficient. In other examples, removing the heart tissue from the helix may include a “saline swish” where the helix is agitated in a bowl of saline, or a “power wash” where saline is forcibly ejected from a syringe onto the helix. In some cases, the saline may fail to dislodge the myocardial tissue and the clinician may use the manual techniques described above.

In some cases, cleaning the helix between re-implantation attempts using manual techniques may take up to 10 minutes per cleaning. On average there are three cleanings of the helix for each procedure. As a result, the cleaning process may cumulatively lengthen a procedure by 30 minutes or more on average. Furthermore, non-specialized (e.g., makeshift) tools may damage the helix which may cause deformation or result in stress concentrations that may result in a fracture years after implantation. Additionally, mechanical friction from improper cleaning methods may detach sensitive components like a drug-eluting collar or deform the structural integrity of the helix itself, rendering the medical lead unusable.

The techniques of this disclosure may facilitate efficient and regulated removal of patient tissue from the helix via a cleaning tool. The cleaning tool may include a frame defining an engagement zone and an engagement element, such as one or more strands, bristles, foam, or any other suitable engagement element suitable to contact patient tissue. An actuation assembly may facilitate a user transitioning the cleaning element from an open configuration to a closed configuration, where the engaging element may mechanically engage the patient tissue. Once engaged, the medical lead may be rotated relative to the cleaning tool to unscrew the helix from the engagement elements in the cleaning configuration. This may remove the patient tissue from the helix while the engagement element may remain stationary. Alternatively, the tool may be rotated relative to the medical lead where the engagement elements may unscrew patient tissue from the helix in the cleaning configuration. This may remove the patient tissue from the stationary helix while the engagement elements may move in a controlled manner about the helix.

The disclosed techniques provide a controlled, repeatable method for cleaning the helix that modulates a maximum force applied to the medical lead. The cleaning tool may protect fragile components, such as drug-eluting rings, from inadvertent contact or excessive force. A rotational rate of the cleaning tool in the cleaning configuration may match a pitch of the helix. A spring may modulate a force applied by an engagement or cleaning element, such as a strand, to the helix. The cleaning tool may provide the advantage of reducing the time required to clean the medical lead compared to manual cleaning which may streamline the implantation procedure and/or may reduce the duration of patient anesthesia. In some examples, utilizing the cleaning tool may reduce a time required to clean the helix to approximately 30 seconds or less. This reduction in cleaning time contrasts with manual methods, which may require several minutes to effectively remove the patient tissue between implantation attempts.

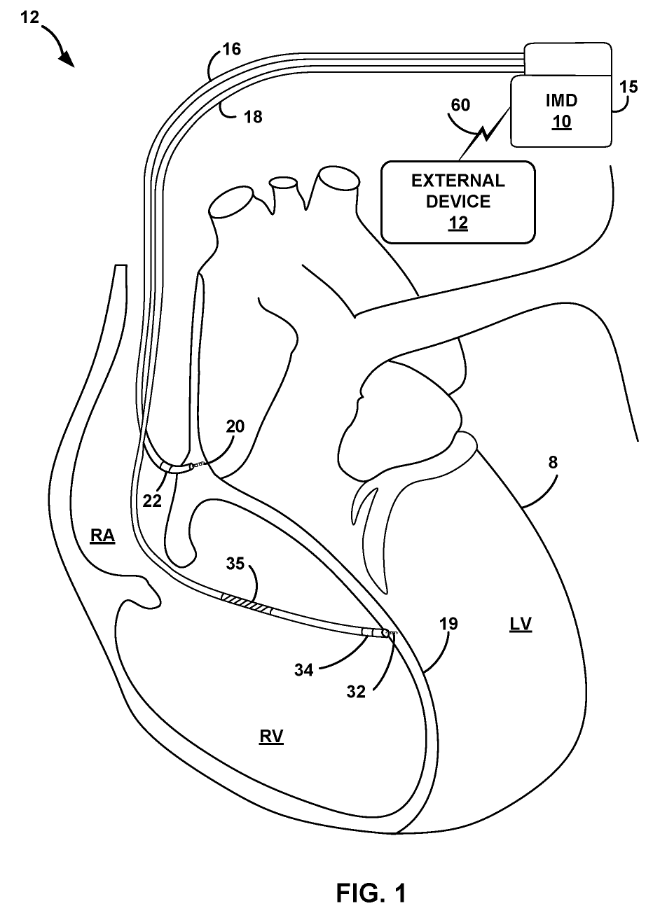

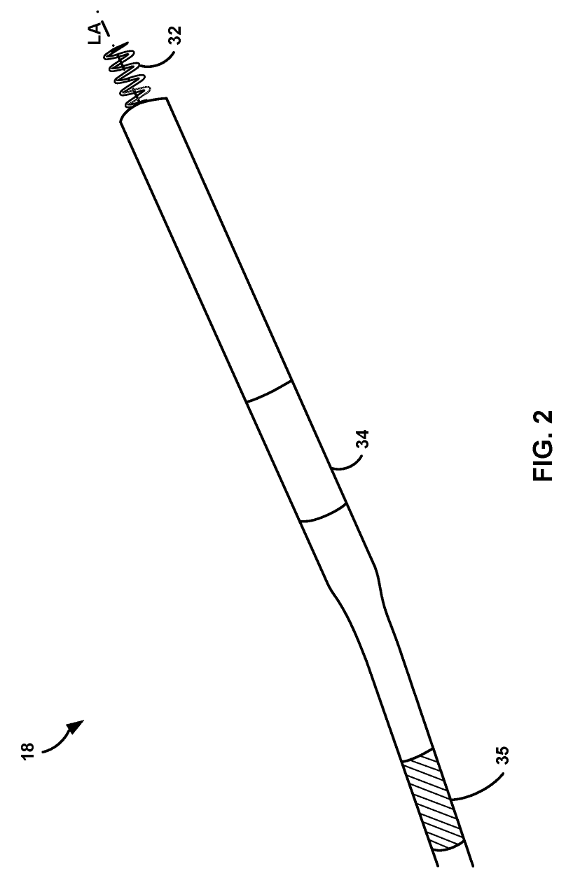

FIG. 1 is a conceptual diagram illustrating an example cardiac system configured to connect to cardiac tissue of a patient. FIG. 2 is a conceptual drawing illustrating a medical lead of FIG. 1 with a helix, in accordance with one or more techniques of this disclosure. FIGS. 1 and 2 are described together for clarity. Medical device system 2 may be capable of pacing and sensing in heart 8 of a patient. Medical device system 2 may include implantable medical device (IMD) 10 and external device 12. IMD 10 may be in wireless communication with at least one of external device 12 and other devices not pictured in FIG. 1. IMD 10 may be implanted within a patient, and coupled to one or more implantable medical leads. IMD 10 may be configured to sense one or more cardiac electrograms (EGMs) and deliver conduction system pacing, such as Left Bundle Branch Area Pacing (LBBAP), via the plurality of electrodes.

External device 12 may be a computing device with a display viewable by the user and an interface for providing input to external device 12 (e.g., a user input mechanism). In some examples, external device 12 may be a notebook computer, tablet computer, workstation, one or more servers, cellular phone, personal digital assistant, or another computing device that may run an application that enables the computing device to interact with IMD 10. In some examples, external device 12 may be implemented as a number of computing systems configured to receive cardiac electrical signals, which may include at least one EGM signal input received directly or indirectly from IMD 10. In some examples, external device 12 is a computing device included in a remote patient monitoring system such as a computing device included in the CARELINK® monitoring system available from Medtronic, Inc., Minneapolis, Minnesota.

External device 12 is configured to communicate with IMD 10 and, optionally, another computing device (not illustrated in FIG. 1), via wireless communication 60. External device 12, for example, may communicate via near-field communication technologies (e.g., inductive coupling, Near-Field Communication (NFC) or other communication technologies operable at ranges less than 10–20 cm) and far-field communication technologies (e.g., Radio Frequency (RF) telemetry according to the 802.11 or Bluetooth® specification sets, or other communication technologies operable at ranges greater than near-field communication technologies).

External device 12 may be used to configure operational parameters for IMD 10. External device 12 may be used to retrieve data from IMD 10. The retrieved data may include values of patient parameters measured by IMD 10, indications of episodes of arrhythmia or other maladies detected by IMD 10, and physiological signals recorded by IMD 10.

IMD 10 may be coupled to heart 8 via transvenous electrical leads 16 and 18. Housing 15 of IMD 10 may enclose internal circuitry corresponding to various circuits and components for sensing cardiac signals from heart 8 and delivering cardiac pacing therapy. In some examples, IMD 10 may be configured to detect tachyarrhythmias from the sensed cardiac signals and deliver high voltage cardioversion/defibrillation (CV/DF) shocks to heart 8, e.g., for terminating a detected ventricular tachycardia or ventricular fibrillation.

Lead 16 may be a Right Atrial (RA) lead (“RA lead 16”) and lead 18 may be a ventricular conduction system (VCS) pacing lead (“VCS pacing lead 18”). RA lead 16 and VCS pacing lead 18 may be advanced transvenously to position electrodes for sensing cardiac electrical signals and delivering pacing therapy. RA lead 16 may be positioned such that its distal end is in, or in the vicinity of, the right atrium. RA lead 16 may carry pacing and sensing electrodes 20 and 22, shown as a tip electrode and a ring electrode, respectively. Electrodes 20 and 22 may provide sensing and/or pacing in the RA. Electrodes 20 and 22 may be connected to a respective insulated conductor extending within the elongated body of RA lead 16. IMD 10 may include a connector block configured to receive a lead connector for electrically coupling conductors extending from the distal electrodes 20 and 22 to circuitry within housing 15 via electrical feedthroughs crossing housing 15.

VCS lead 18 may be advanced from the right atrium to the right ventricle to position tip electrode 32 and ring electrode 34 for pacing and/or sensing in the vicinity of the VCS, e.g., at or near the left bundle branch (LBB), from a right atrial approach, as shown. VCS tip electrode 32 may be a helical electrode. VCS tip electrode 32 may be advanced into the inferior end of the interatrial septum, beneath the atrioventricular (AV) node and near the tricuspid valve annulus to position tip electrode 32 in, along or proximate to the His bundle. Ring electrode 34 spaced proximally from tip electrode 32 may be used as the return electrode with the cathode tip electrode 32 for pacing the right and left ventricles via the native His-Purkinje conduction system.

IMD 10 may produce an intracardiac EGM signal from the cardiac electrical signal received via a sensing electrode vector that may include tip electrode 32 and/or ring electrode 34 of VCS lead 18. Electrodes 32 and 34 may be coupled to respective insulated conductors extending within the elongated body of VCS lead 18, which provide electrical connection to a proximal lead connector coupled to circuitry of IMD 10 within housing 15 via electrical feedthroughs crossing housing 15.

Housing 15 may function as a return electrode for unipolar sensing and/or pacing configurations with any of the electrodes carried by leads 16 and 18. Electrodes 32 and 34 may be used in a bipolar pacing electrode pair for delivering VCS pacing pulses and/or for receiving a cardiac electrical signal for sensing intrinsic and pacing evoked QRS waveforms. In some examples, IMD 10 may be configured to sense a far field (FF) cardiac signal, e.g., using electrode 32 and housing 15 or using electrode 34 and housing 15. In other examples, IMD 10 may be configured to sense near field (NF) cardiac signal, e.g., using electrodes 32 and 34. Electrodes 32 and 34 may be used in a sensing electrode vector for sensing intrinsic R-waves for use in determining a heart rhythm and a need for electrical stimulation therapy.

In FIG. 1, IMD 10 is a dual chamber device configured to receive RA lead 16, positioned in the right atrial chamber for delivering atrial pacing pulses and sensing atrial electrical signals via electrodes 20 and 22. IMD 10 may be configured to sense intrinsic atrial P-waves and deliver atrial pacing pulses in the absence of sensed P-waves. IMD 10 may be configured to deliver atrial synchronized ventricular pacing by setting an AV delay in response to each sensed P-wave or delivered atrial pacing pulse and deliver a ventricular pacing pulse to the VCS via lead 18 upon the expiration of the AV delay.

VCS lead 18 may be advanced transvenously into the right ventricle (RV) via the RA for positioning tip electrode 32 within inter-ventricular septum 19. Tip electrode 32 may be selected as a pacing cathode electrode in combination with ring electrode 34 as the return anode electrode for pacing and capturing the LBB and/or right bundle branch (RBB) in various examples. In some instances, the pacing magnitude (e.g., pulse amplitude and/or pulse width) may be selected to achieve cathodal capture at the cathode electrode for capturing at least one bundle branch. In some instances, the pacing pulse amplitude and/or pulse width may be selected to achieve cathodal and anodal capture, which may capture the LBB and the right side of the ventricular septum. In some instances, the LBB and the RBB may be captured concurrently to provide dual or bilateral bundle branch (BB) pacing using a single bipolar electrode pair.

In other examples, either tip electrode 32 or ring electrode 34 may be selected as a cathode paired with housing 15 in a unipolar pacing electrode vector. Unipolar pacing may capture a single BB or location within the ventricles. While VCS lead 18 is shown carrying one pacing and sensing electrode pair, e.g., tip electrode 32 and ring electrode 34, in other examples VCS lead 18 may include multiple electrodes. Including multiple electrodes may provide the advantage of enabling one or more selectable bipolar pacing electrode vectors and/or one or more unipolar pacing electrode vectors (e.g., with housing 15) for delivering pacing pulses to the left and right side of the ventricular septum. VCS lead 18 may include one or more coil electrodes, e.g., coil electrode 35, when IMD 10 is configured as an ICD capable of delivering high voltage shock therapies. Coil electrode 35 may be used in sensing electrode vectors, e.g., with either of tip electrode 32 or ring electrode 34.

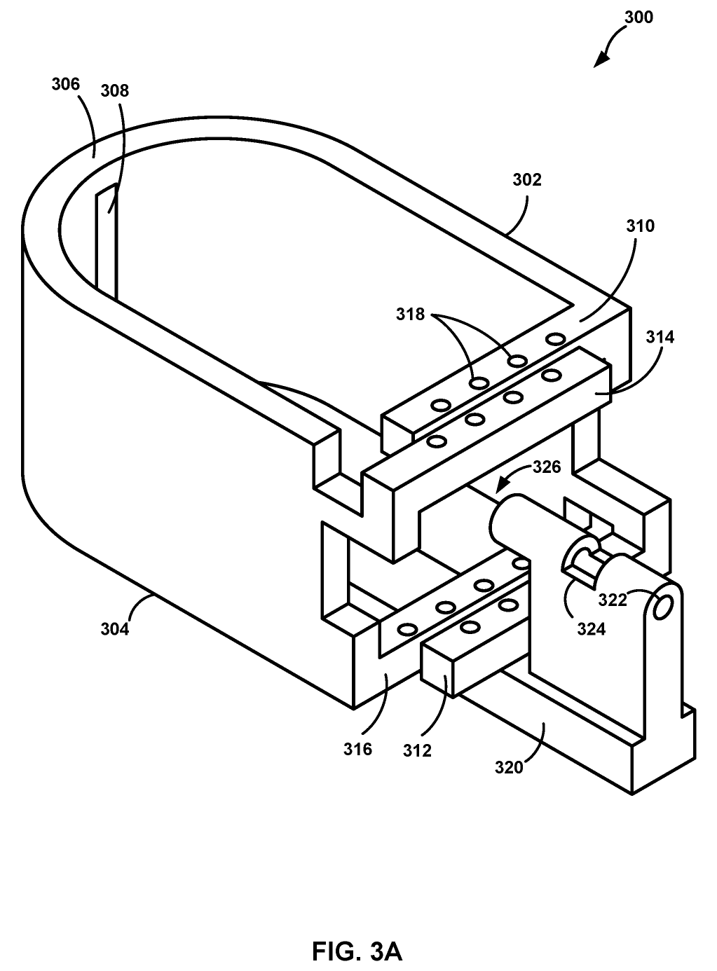

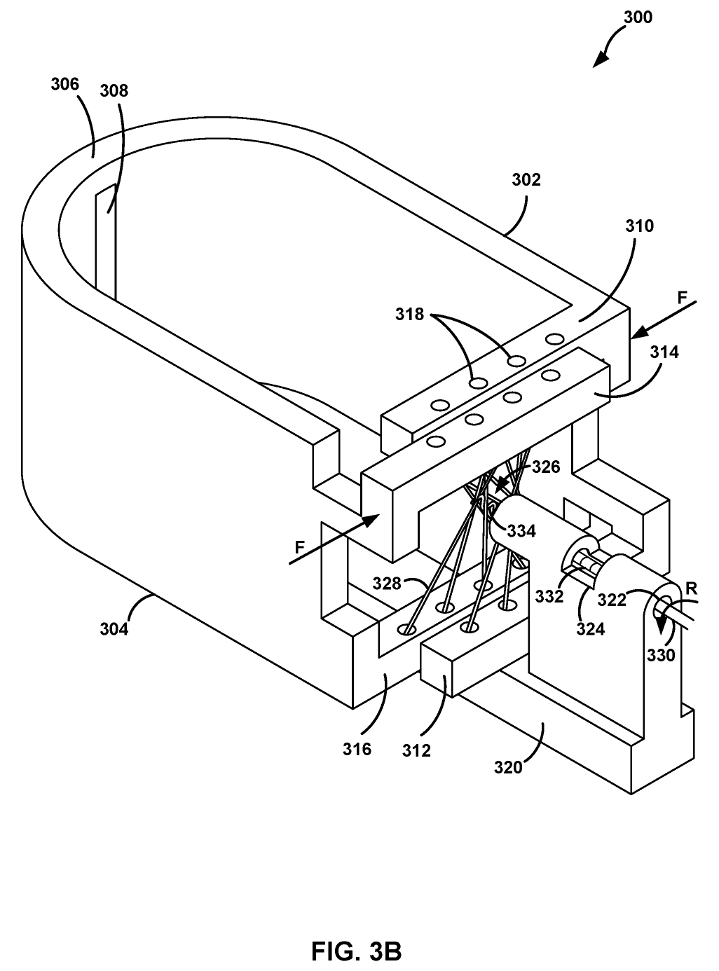

FIG. 3A is a perspective view illustrating an example cleaning device in a loading configuration without strands, in accordance with one or more techniques of this disclosure. FIG. 3B is a perspective view illustrating the example cleaning device of FIG. 3A in a cleaning configuration with strands engaging a helix of a medical lead. FIGS. 3A and 3B are described together for clarity.

Cleaning device 300 may be configured to remove patient tissue from a helix of a medical lead. Cleaning device 300 may include first frame portion 302 and second frame portion 304 coupled at pivot 306. Pivot 306 may be an actuation element, e.g., actuation assembly, which may enable first frame portion 302 and second frame portion 304 to transition from a first configuration to a second configuration. First frame portion 302 and second frame portion 304 may pivot relative to one another about pivot 306, functioning in a manner similar to a pair of tweezers, forceps, or pliers. In the example shown, pivot 306 may be a living hinge formed integrally with the first frame portion 302 and the second frame portion 304. In other examples, pivot 306 may be a mechanical pin joint, a ball-and-socket joint, a flexure bearing, or another suitable coupling mechanism that enables relative movement. In other examples, pivot 306 may be a scissor mechanism located intermediate between a proximal handle portion and a distal engagement zone, such that compressing the handle portion closes the engagement zone. In other examples, first frame portion 302 and second frame portion 304 may utilize a linear slider mechanism, where first frame portion 302 and second frame portion 304 are coupled via a track or rail system to enable linear translation relative to one another to clamp helix 334.

First frame portion 302 and second frame portion 304 may be defined by a U-shape. In other examples, first frame portion 302 and second frame portion 304 are defined by a C-shape, a V-shape profile, or any other profile suitable for compression by a user. First frame portion 302 and second frame portion 304 may be constructed from various materials, such as injection-molded plastics (e.g., polycarbonate, Acrylonitrile Butadiene Styrene (ABS)), medical-grade metals (e.g., stainless steel, titanium), or composite materials. In some examples, cleaning device 300 may be configured as a single-use, disposable unit intended to be discarded after the procedure. First frame portion 302 and/or second frame portion 304 may include a reuse prevention feature such as a frangible seal that breaks upon initial actuation, or a non-reversible locking tab. The reuse prevention feature may reduce re-opening or re-closing of first frame portion 302 and/or second frame portion 304. The re-use prevention feature may provide the advantage that cleaning device 300 is configured to avoid cleaning device 300 from being be improperly sterilized and reused. In other examples, cleaning device 300 may be constructed from materials compatible with a sterilization process to facilitate reuse of the device in multiple procedures. In some examples, the sterilization process may include autoclaving, chemical sterilization, or any other suitable sterilization process. The materials compatible with a sterilization process may include polyetheretherketone (PEEK), polysulfone (PSU), polyethersulfone (PES), polytetrafluoroethylene (PTFE), titanium, stainless steel, and cobalt chrome.

Cleaning device 300 further includes an engagement assembly defined by a plurality of arms extending from first frame portion 302 and second frame portion 304. The plurality of arms includes first frame portion 302 which may include inner top arm 310 and outer bottom arm 312 and second frame portion 304 which may include outer top arm 314 and inner bottom arm 316. Inner top arm 310 and outer top arm 314 may extend generally parallel to one another on a first side of engagement zone 326, while outer bottom arm 312 and inner bottom arm 316 may extend generally parallel to one another on a second, opposing side of engagement zone 326. In other examples, the plurality of arms may be angled, curved, or tapered to facilitate visibility or ergonomic handling. The plurality of arms (e.g., inner top arm 310, outer bottom arm 312, outer top arm 314, and inner bottom arm 316) may each define a plurality of strand slots 318 configured to anchor one or more strands 328. Strand slots 318 may be molded grooves, laser-cut notches, through-holes, or raised posts.

Engagement zone 326 is a space defined between the plurality of arms configured to receive a distal portion of lead 330. As shown in FIG. 3B, cleaning device 300 may further include lead chuck 320 configured to stabilize lead 330. In some examples, lead 330 may be a pacing lead configured to electrically connect an implantable pulse generator, which may be implanted pectorally, to a heart of the patient. In other examples, lead 330 may also refer to a lead configured for implantation at other locations in the patient, e.g., non-cardiac locations, and may include a fixation mechanism other than a helix cleaned according to the techniques described herein. In some examples, a helix of the leadless pacemaker or other leadless stimulator or monitor may be cleaned utilizing the devices and techniques described herein. Lead 330 may include a Monolithic Controlled Release Device (MCRD) ring which may remain proximal to engagement zone 326. Maintaining the MCRD ring proximal may enable strands 328 to avoid abrading or detaching the MCRD ring during the cleaning process.

Lead chuck 320 defines lead slot 322 sized to receive a lead body of lead 330. Lead slot 322 may include a friction material, a rubber lining, or a textured surface to enhance grip on lead 330. Lead chuck 320 may also define alignment window 324 positioned to provide visual confirmation of a position of lead 330. For example, alignment window 324 may be positioned such that lead feature 332 is visible through the alignment window 324 when a helix 334 of the lead 330 is properly positioned within the engagement zone 326. Lead feature 332 may include a ring electrode, a visual marker band, or a specific transition in lead diameter. In some examples, lead feature 332 is an anode ring of medical lead 330. Lead chuck 320 may define a plurality of alignment windows 324 spaced longitudinally along lead slot 322. This arrangement may enable cleaning device 300 accommodate a variety of different medical lead models having different spacing between helix 334 and the anode ring.

The user may select the appropriate alignment window 324 corresponding to the specific model of medical lead 330 being cleaned. This alignment may provide the advantage of facilitating confirmation that helix 334 is positioned for cleaning while other components of lead 330, such as a drug-eluting collar or a sensing electrode, remain clear of engagement zone 326 to avoid damage. Alignment window 324 may be an open aperture, a slot, or a transparent window formed from a clear polymer or glass. The use of lead feature 332 (e.g., the ring electrode) may provide the advantage of improved alignment if helix 334 is obscured. For example, if helix 334 is heavily encrusted with patient tissue, helix 334 may be obscured, making direct visual alignment with engagement zone 326 difficult. Proximal lead feature 332 may be less obscured which may improve visual alignment to properly position the obscured helix 334 within engagement zone 326 without visual confirmation of the position of helix 334 itself.

One or more strands 328 are coupled to the frame portions and span across engagement zone 326. In some examples, strands 328 may include a first strand set and a second strand set. The first strand set may span from inner top arm 310 to outer bottom arm 312. The second strand set may span from outer top arm 314 to inner bottom arm 316. This arrangement creates a crisscross or intersecting pattern of strands 328 within the engagement zone 326. This opposing arrangement of the first strand set and the second strand set is configured to apply a radially balanced force to the helix 334 when in the cleaning configuration. By engaging helix 334 from opposing sides, strands 328 may maintain helix 334 on a central axis of cleaning device 300, reducing off-axis bending moments that might otherwise deform helix 334 if a single unilateral strand were used.

In other examples, strands 328 may be arranged in a parallel pattern, a mesh pattern, a woven net, or a star pattern. In some examples, strands 328 may include a monofilament suture configured to provide a smooth, non-abrasive cleaning surface. In other examples, strands 328 may include tethers. Strands 328 may be configured to operate in a dry state, without the application of saline or lubrication. Operating cleaning device 300 in a dry state may increase the coefficient of friction between strands 328 and the patient tissue on helix 334, facilitating the mechanical shearing of the tissue. Alternatively, engagement zone 326 may be flushed with a fluid if lubricating the surface of helix 334 is desired to further protect a coating on lead 330.

In other examples, strands 328 may be formed from a biocompatible material, such as a suture material, a monofilament line, a braided thread, a nylon fiber, or a metallic wire. In other examples, strands 328 may include a braided suture material having a textured surface configured to increase mechanical friction against patient tissue for enhanced removal. The specific material and tension of strands 328 may be selected to be stiff enough to remove tissue from helix 334 but flexible enough to avoid damaging a surface or coating of helix 334. Furthermore, the coupling of strands 328 to strand slots 318 may enable a degree of movement. This degree of movement may enable the strands 328 to self-align and “walk” with the coils of helix 334 as lead 330 is rotated, thereby maintaining consistent engagement with the helical geometry without binding or applying excessive off-axis force.

FIG. 3A illustrates the cleaning device 300 in a first, loading configuration. In this state, no external force is applied to first frame portion 302 or second frame portion 304, and the plurality of arms are spaced apart such that strands 328 (not shown in FIG. 3A for clarity) are relatively loose or define an open aperture large enough to receive helix 334 without interference. In the loading configuration, first frame portion 302 and second frame portion 304 may define an angle of 15 degrees to 35 degrees relative to a closed axis. The angle may provide sufficient clearance for helix 334 to enter engagement zone 326 without contacting strands 328, reducing the risk of premature engagement or snagging. A user may insert lead 330 into lead slot 322 and slide lead 330 distally until lead feature 332 aligns with alignment window 324, positioning helix 334 within engagement zone 326.

FIG. 3B illustrates cleaning device 300 in a second, cleaning configuration. To transition to this state, a user may apply a compressive force F to first frame portion 302 and second frame portion 304. This force may pivot first frame portion 302 and second frame portion 304 about pivot 306 which may result in inner top arm 310 sliding past outer top arm 314 and outer bottom arm 312 sliding past inner bottom arm 316. This relative motion alters the location of strand slots 318 which may tighten strands 328 and may cause the crisscross pattern to constrict around helix 334. The arrangement of strands 328 may enable them to engage between the coils of helix 334. The relative amount of compressive force F may correlate to the relative amount of movement of strands 328 which may provide the advantage of enabling the user to modulate compressive force F to modulate tension applied to helix 334 by strands 328. This variable tensioning may provide the advantage of facilitating the user to modulate the cleaning force based on the amount of debris, the helix 334 being cleaned, or any other suitable factors contemplated by the user which may include a force to clean helix 334.

While maintaining compressive force F to keep strands 328 engaged with helix 334, the user rotates lead 330 in a direction R (e.g., counter-clockwise). This rotation causes helix 334 to unscrew from the engagement with strands 328. As helix 334 retracts proximally out of engagement zone 326, strands 328 frictionally engage and immobilize any patient tissue present on helix 334. In other words, strands 328 frictionally engage and immobilize the patient tissue to effectively strip the patient tissue from the helix of the lead. Cleaning device 300 may include a mechanical stop, such as frame slot 308 or a tab feature on the arms, configured to limit the range of motion of first frame portion 302 relative to second frame portion 304. This mechanical stop may provide the advantage of avoiding over-compression of strands 328 against helix 334 which may provide the advantage of protecting helix 334 from deformation or surface damage. Other examples of mechanical stops may include a set screw, a molded ridge, or a deformable bumper.

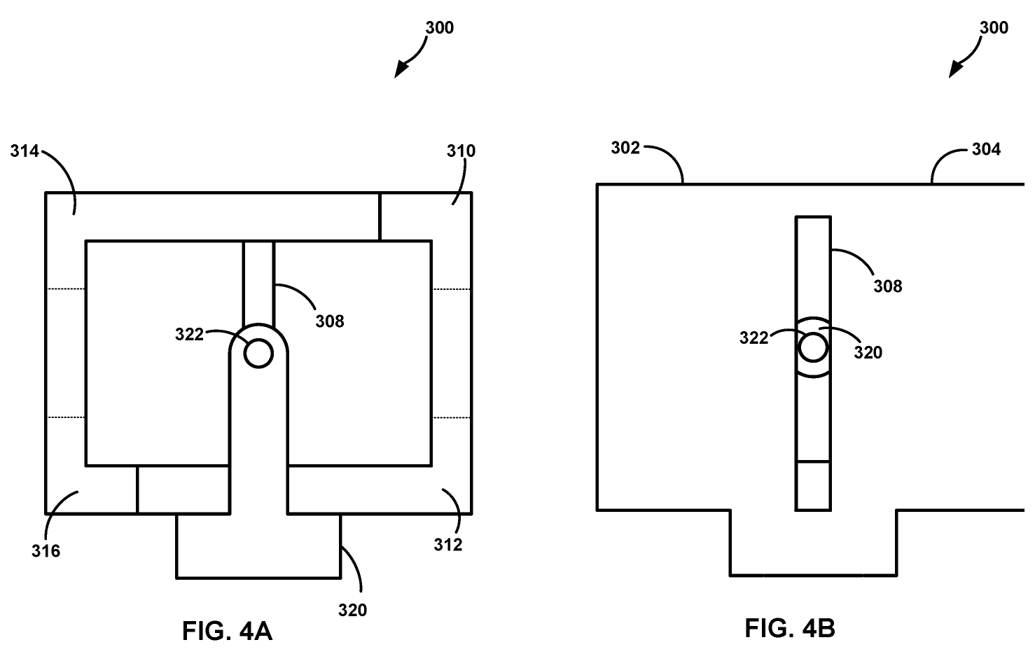

FIG. 4A is a front view of cleaning device 300 of FIG. 3A. FIG. 4B is a back view illustrating the example frame. FIGS. 4A and 4B are described together for clarity.

Cleaning device 300 may include first frame portion 302 and second frame portion 304. First frame portion 302 may include inner top arm 310 and outer bottom arm 312. Second frame portion 304 may include outer top arm 314 and inner bottom arm 316.

Cleaning device 300 may further include lead chuck 320 located at a base of the frame. Lead chuck 320 may define lead slot 322 configured to receive the medical lead. In the example shown in FIG. 4B, lead slot 322 may extend through lead chuck 320 to enable the lead to pass entirely through the device. Lead slot 322 may have a circular cross-section, a U-shaped cross-section, or a V-shaped cross-section to accommodate leads of varying diameters (e.g., 4 French, 7 French). In some examples, lead slot 322 may be dimensioned to accommodate medical leads having diameters of 4.1 French or 4.7 French. In other examples, the lead slot 322 may be sized to receive a range of lead diameters, such as from 3 French to 7 French, to facilitate compatibility with various conduction system pacing leads. In other examples, lead chuck 320 may include a locking mechanism, such as a snap-fit latch or a sliding gate, to secure the lead within lead slot 322 during the cleaning process.

First frame portion 302 and second frame portion 304 may define frame slot 308. Frame slot 308 may serve as a guide or a clearance channel that facilitates the pivoting or sliding motion of first frame portion 302 relative to second frame portion 304. Additionally, frame slot 308 may function as a range-of-motion limiter. For example, surfaces of first frame portion 302 may contact ends of frame slot 308 when the device reaches a maximum open position or a maximum closed position. This limitation may provide the advantage of avoiding over-extension of the frame or excessive crushing force applied to the helix by the engagement assembly. Other examples of motion limiters may include external hard stops, internal pins, or interfering ribs molded into the frame portions.

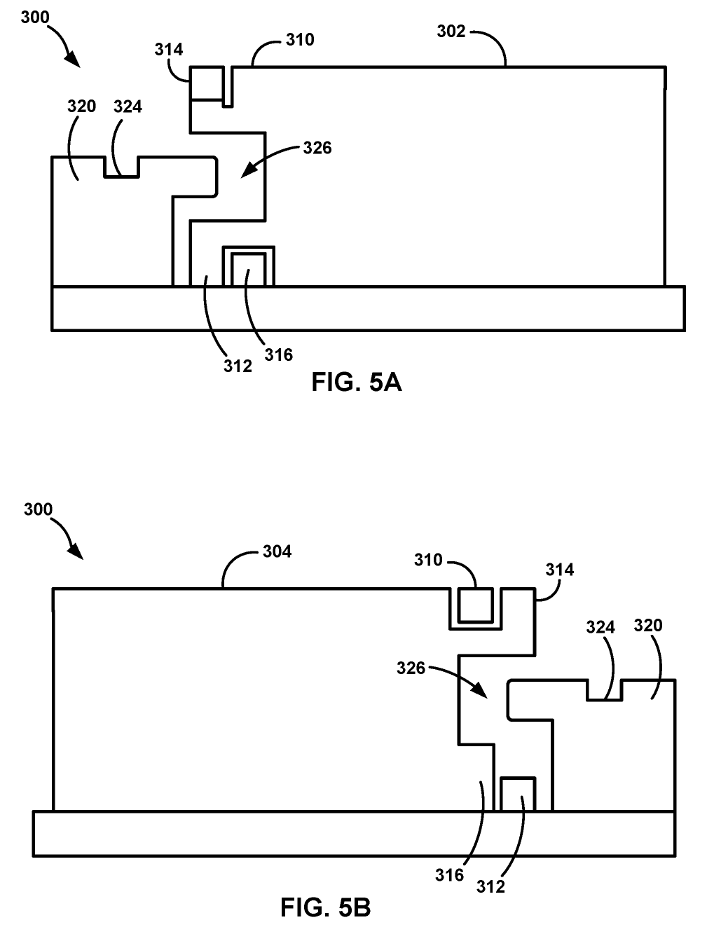

FIG. 5A is a first side view illustrating a first frame portion of the example cleaning device of FIG. 3A. FIG. 5B is a side elevation view illustrating a second frame portion of the example cleaning device of FIG. 3A. FIGS. 5A and 5B are described together for clarity.

As shown in FIG. 5A, first frame portion 302 includes an inner top arm 310 and an outer bottom arm 312. As shown in FIG. 5B, the second frame portion 304 includes an outer top arm 314 and an inner bottom arm 316. Lead chuck 320 may be integrally formed with first frame portion 302 and/or second frame portion 304, or it may be a separate component coupled to first frame portion 302 and/or second frame portion 304 via a snap-fit, adhesive, or welded connection. Lead chuck 320 may define alignment window 324. Alignment window 324 may enable a user to visualize the medical lead passing through lead chuck 320. This visibility may provide the advantage of facilitating the user aligning a feature of the lead with alignment window 324 which may facilitate positioning of the helix in the engagement zone.

Inner top arm 310, outer bottom arm 312, outer top arm 314, and inner bottom arm 316 may define engagement zone 326. Inner top arm 310, outer bottom arm 312, outer top arm 314, and inner bottom arm 316 may be configured such that inner top arm 310 fits adjacent to outer top arm 314 and inner bottom arm 316 fits adjacent to outer bottom arm 312. This may provide the advantage of creating a compact device profile while engagement zone 326 remains centrally located relative to lead chuck 320 during the actuation of the frame portions. This may additionally or alternatively provide the advantage of creating a crisscross pattern which may further increase an engagement by the strands with the patient tissue.

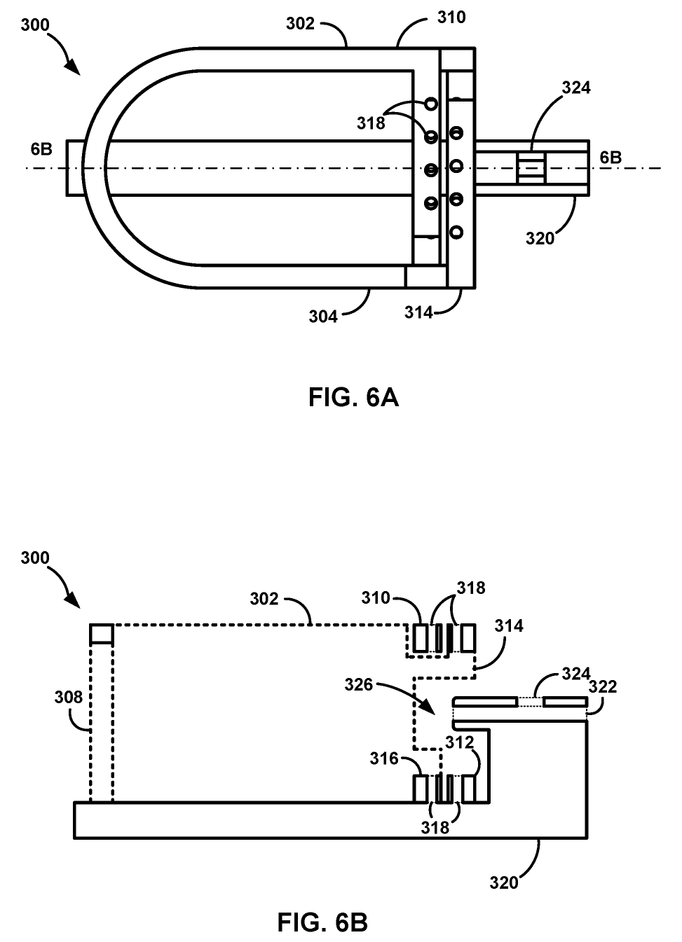

FIG. 6A is a top plan view illustrating the example cleaning device of FIG. 3A. FIG. 6B is a cross-sectional view of the example cleaning device frame taken along line 6B-6B of FIG. 6A. FIGS. 6A and 6B are described together for clarity.

Cleaning device 300 may include first frame portion 302 and second frame portion 304. First frame portion 302 may include inner top arm 310 and outer bottom arm 312. Second frame portion 304 may include outer top arm 314 and inner bottom arm 316. Frame slot 308 may separate the actuation ends of first frame portion 302 and second frame portion 304, enabling relative compression.

As shown in FIG. 6A, inner top arm 310, outer bottom arm 312, outer top arm 314, and inner bottom arm 316 each define a plurality of strand slots 318. In the example shown, strand slots 318 are depicted as through-holes extending vertically through the arms. In other examples, strand slots 318 may be formed as lateral notches, molded hooks, posts, cleats, or any other suitable shape configured to secure a strand. The arrangement of strand slots 318 facilitates the threading of engagement strands (not shown) across engagement zone 326. For example, a strand may be threaded from strand slot 318 on inner top arm 310 to a corresponding strand slot 318 on outer bottom arm 312, creating a diagonal span across engagement zone 326. This configuration may provide the advantage of creating a multi-dimensional cleaning matrix that contacts the helix from multiple angles when the arms are compressed.

Lead chuck 320 may define lead slot 322 extending along a longitudinal axis of the device. Lead slot 322 may be a slot sized to slidably receive the medical lead. Lead chuck 320 may further define alignment window 324 intersecting lead slot 322. As shown, alignment window 324 may be a cutout or aperture enabling visual access to the interior of lead slot 322. Positioning an alignment feature in alignment window 324 may enable a user to verify that the helix has fully entered engagement zone 326 while facilitating a drug-eluting collar or other sensitive feature remains protected within the shielded region of lead slot 322 proximal to alignment window 324. In other examples, alignment window 324 may include a magnifying lens or a colored background to enhance visibility of the lead feature.

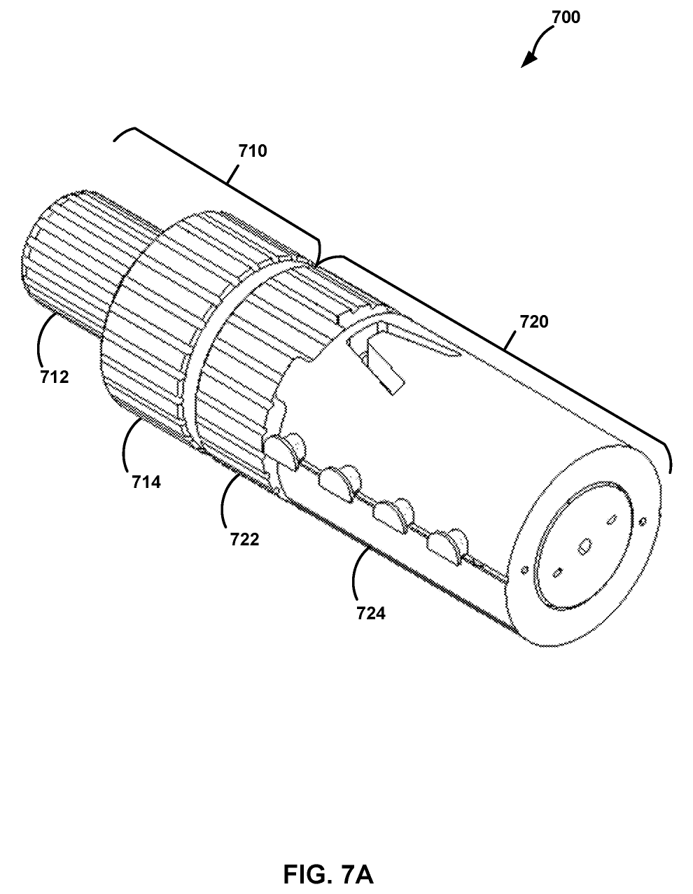

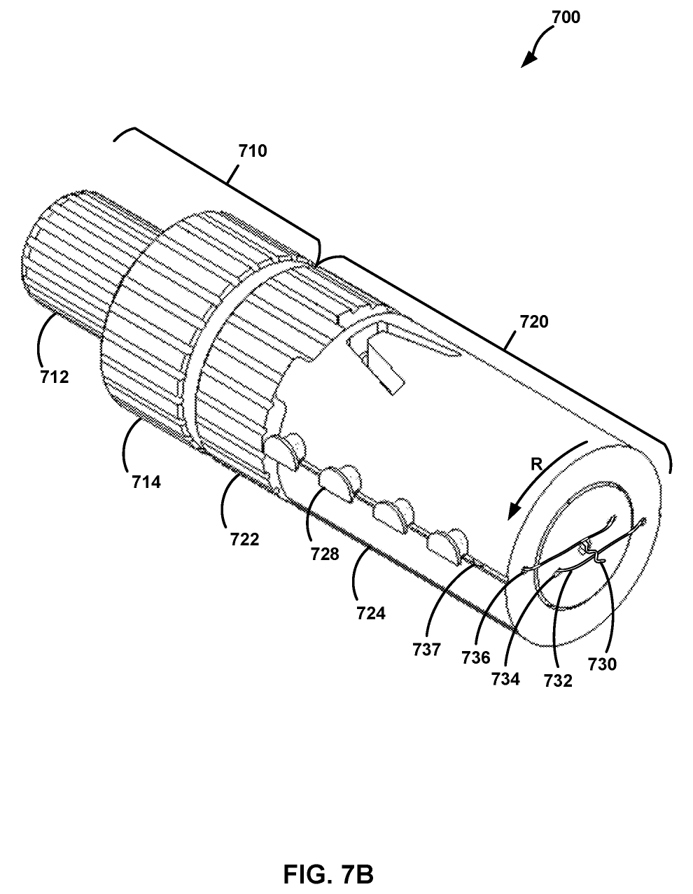

FIG. 7A is a perspective view illustrating an example cleaning device in a loading configuration. FIG. 7B is a perspective view illustrating the example cleaning device of FIG. 7A with strands in the loading configuration around the helix. FIGS. 7A and 7B are described together for clarity. Cleaning device 700 may include lead retention chuck 710 and actuation assembly 720. Actuation assembly 720 may perform a controlled wrapping and withdrawal sequence to clean helix 730.

Lead retention chuck 710 may releasably secure a distal portion of a medical lead. Lead retention chuck 710 may include lead retention sleeve 712 and chuck tightener 714. Lead retention sleeve 712 may define a collet, a compressible lumen, or a deformable interface sized to receive the medical lead. Rotation of chuck tightener 714 relative to lead retention sleeve 712 (or vice versa) may radially compress lead retention sleeve 712 against a lead body. This clamping action may provide the advantage of gripping the lead body relatively close to a distal tip. Gripping near the distal tip may reduce torsional energy buildup (e.g., “coiling”) within the lead body during rotation of actuation assembly 720. Lead retention sleeve 712 may utilize a polymer, rubber, or silicone material to avoid damage to lead insulation.

Actuation assembly 720 may couple to lead retention chuck 710 and extend distally to define a chamber for receiving helix 730. Actuation assembly 720 may include cleaning actuator 722 and wrapping actuator 724. Cleaning actuator 722 may be an inner cylindrical component coupled to lead retention chuck 710. Wrapping actuator 724 may be an outer shell rotatable relative to cleaning actuator 722. Actuation assembly 720 supports one or more strands 732. One or more strands 732 may comprise two strands 732 running parallel to each other to balance force on helix 730. Strands 732 may connect to an optional spring (not shown) connected to cleaning actuator 722. Alternatively, strands 732 may connect directly to cleaning actuator 722. Strands 732 may run axially through cleaning actuator 722 and exit cleaning strand hole 734. Strands 732 may then extend along an axial face of cleaning actuator 722 and wrapping actuator 724 to first wrapping strand hole 736. In a loading configuration, strands 732 may be straight in a loading configuration as illustrated in FIG. 7B. Alternatively, strands 732 may wrap around helix 730 in a cleaning configuration (not shown). Strands 732 may extend from first wrapping strand hole 736 through wrapping actuator 724 to second wrapping strand hole 737. Strands 732 may be secured to cleaning grip 728 (e.g., by tying). As illustrated, wrapping actuator 724 may include four cleaning grips 728 which may define four positions to secure strands 732. Securing strands 732 to cleaning grips 728 distally from second wrapping strand hole 737 may increase a tension on helix 730.

FIG. 7B illustrates cleaning device 700 in the loading configuration with helix 730 inserted. In this state, wrapping actuator 724 may position strandsb to define an open aperture facilitating insertion of helix 730 without interference. A user may rotate wrapping actuator 724 relative to cleaning actuator 722 (e.g., in direction R) to transition from a loading configuration to a cleaning configuration. Rotation of wrapping actuator 724 may move first wrapping strand hole 736 relative to cleaning strand hole 734. Relative movement causes strands 732 to wrap around helix 730. Strands 732 may wrap around helix 730 from opposite directions to apply a radially balanced force. This wrapping action may create a controlled engagement or “knot” of strands 732 around coils of helix 730. A mechanical stop may define a starting point and an ending point for rotation of wrapping actuator 724 relative to cleaning actuator 722. The optional spring may modulate a tension applied to helix 730 to not exceed a threshold that could deform helix 730 or damage a coating.

Once strands 732 wrap around helix 730 and engage patient tissue, a user may operate cleaning actuator 722 to remove debris. Cleaning actuator 722 may couple to lead retention chuck 710 via a threaded interface. Upon engaging the mechanical stop, further rotation of wrapping actuator 724 may drive rotation of cleaning actuator 722 relative to lead retention chuck 710. Rotation of cleaning actuator 722 relative to lead retention chuck 710 may cause actuation assembly 720 to move longitudinally away from lead retention chuck 710. The threaded interface may define a thread pitch matching a pitch of helix 730. Consequently, as wrapping actuator 724 is rotated, helix 730 may unscrew from engaged strands 732 at a rate matching a natural pitch of helix 730. This synchronized rotation and withdrawal may provide an advantage of stripping away tissue without stretching, compressing, or bending helix 730.

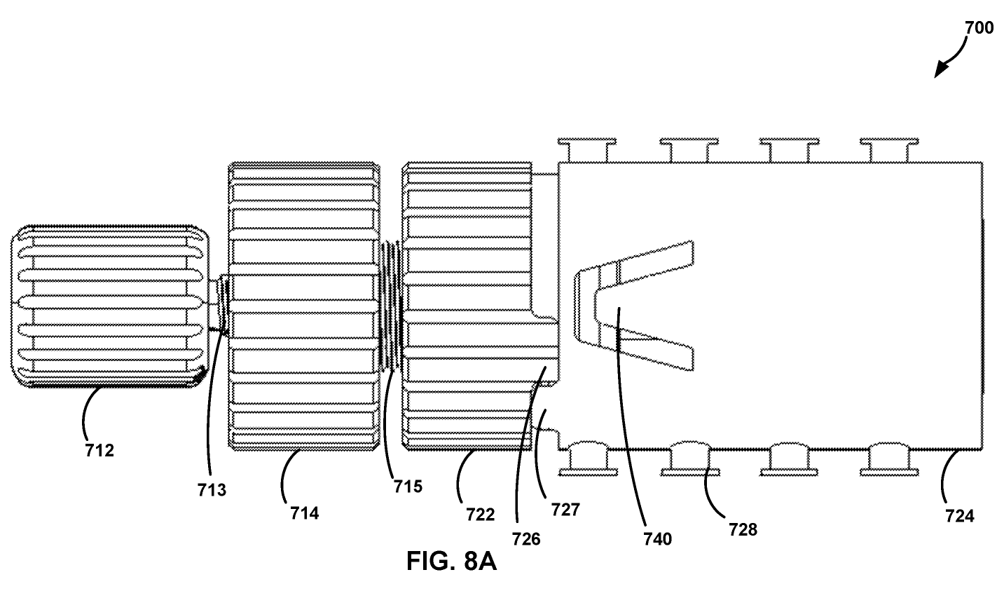

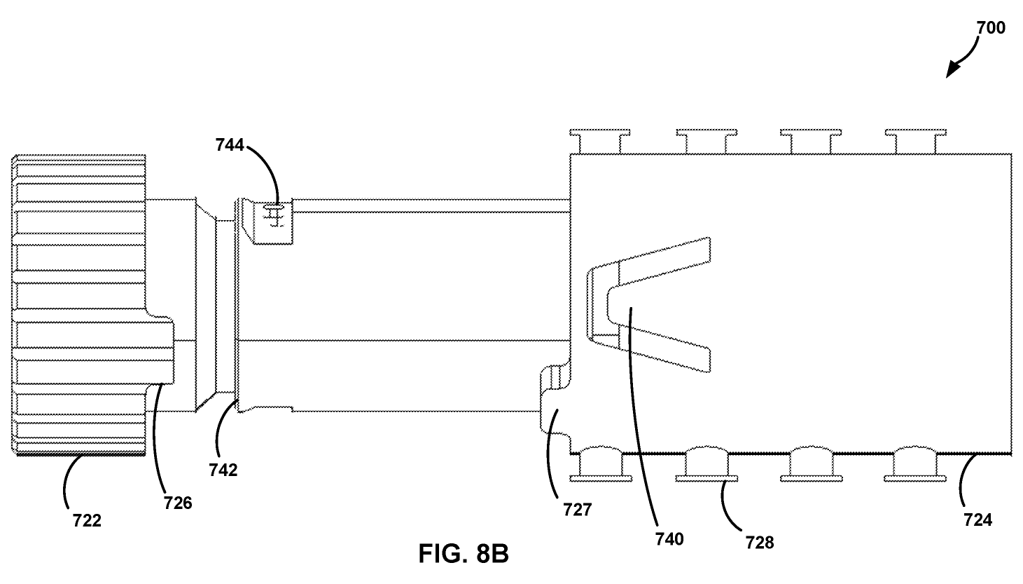

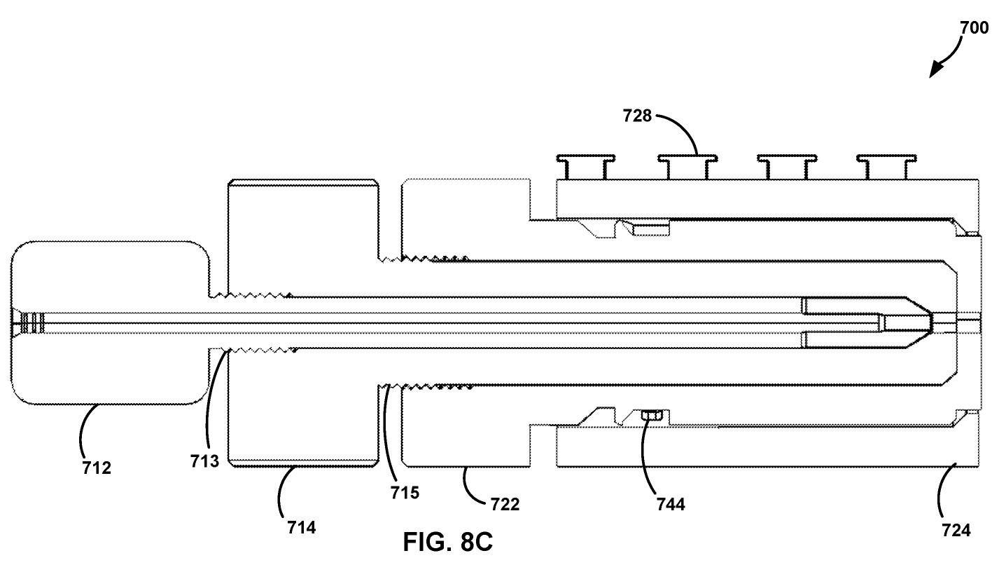

FIG. 8A is a side view illustrating example cleaning device 700 of FIG. 7A. FIG. 8B is an exploded side view illustrating the example cleaning device of FIG. 8A. FIG. 8C is a cross-sectional view of the example cleaning device of FIG. 8A. FIGS. 8A, 8B, and FIG. 8C are described together for clarity.

Lead retention sleeve 712 and chuck tightener 714 may define a lead retention chuck. Cleaning actuator 722 and wrapping actuator 724 may define an actuation assembly. The actuation assembly may be referred to as a cleaning assembly. The actuation assembly may be positioned distal and radially outwards of the lead retention chuck. Cleaning device 700 may include lead retention sleeve 712, chuck tightener 714, cleaning actuator 722, and wrapping actuator 724.

Lead retention sleeve 712 may include sleeve threaded interface 713 configured to engage chuck tightener 714. Rotational action along sleeve threaded interface 713 compresses the collet portion of lead retention sleeve 712 to secure the medical lead within the device. Sleeve threaded interface 713 may be a standard machine thread, a coarse thread, or a tapered thread. Chuck tightener 714 and cleaning actuator 722 define threaded interface 715. Threaded interface 715 may define an actuation assembly to transition the engagement element (e.g., one or more strands with cleaning actuator 722) from a first configuration to a second configuration. Threaded interface 715 may couple chuck tightener 714 to cleaning actuator 722. A pitch of threaded interface 715 may match a pitch of a helix (e.g., helix 730). Actuation along threaded interface 715 may longitudinally displace the lead retention chuck and the medical lead relative to the actuation assembly. For example, rotation of cleaning actuator 722 along threaded interface 715 may longitudinally displace cleaning actuator 722 away from chuck tightener 714. One or more strands engaging biological material may be connected to cleaning actuator 722. As cleaning actuator 722 longitudinally and rotationally displaces, the one or more strands may carry the biological material off of the helix. Matching the pitch of the helix to the pitch of threaded interface 715 may provide the advantage of enabling cleaning actuator 722 and the one or more strands to track the helix as threaded interface 715 is actuated, which may reduce stress and strain on the helix.

Cleaning actuator 722 may act as a base for the actuation assembly. Cleaning actuator 722 may include cleaning mechanical stop 726. Wrapping actuator 724 may be rotatably coupled to cleaning actuator 722 and may include wrapping mechanical stop 727. Cleaning mechanical stop 726 and wrapping mechanical stop 727 may be configured to physically interface with one another to limit a degree of rotation of wrapping actuator 724 relative to cleaning actuator 722. For example, the stops may define a rotational range of approximately 90 degrees to 360 degrees. This rotational limitation may provide the advantage of avoiding over-tightening of the strands against the helix, thereby protecting the helix from deformation. Wrapping actuator 724 may further include cleaning grip 728, such as a series of ridges, knurling, or a rubberized coating, to facilitate manual rotation by a user.

Wrapping actuator 724 may include retention features to reversibly connect to cleaning actuator 722. Wrapping actuator 724 may include snap joint hook 740, which may be a flexible cantilevered arm with a retention barb. Cleaning actuator 722 may define snap joint recess 742, such as a circumferential groove or a track. Snap joint hook 740 may engage snap joint recess 742 to mechanically couple wrapping actuator 724 to cleaning actuator 722 while permitting relative rotation. Additionally, cleaning actuator 722 or wrapping actuator 724 may include spring hook 744. Spring hook 744 may be a small post, eyelet, or anchor point located within the internal chamber of the device. Spring hook 744 may be configured to anchor a biasing element, such as a tension spring (not shown), which may be configured to maintain a tension on the strands during the cleaning process. In other examples, the strands may be directly connected to spring hook 744.

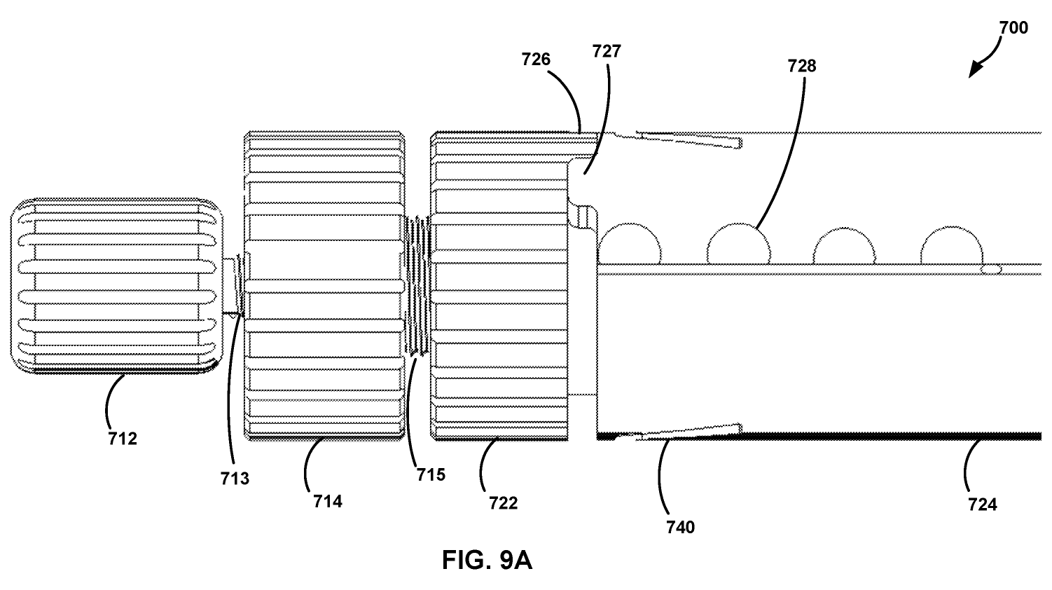

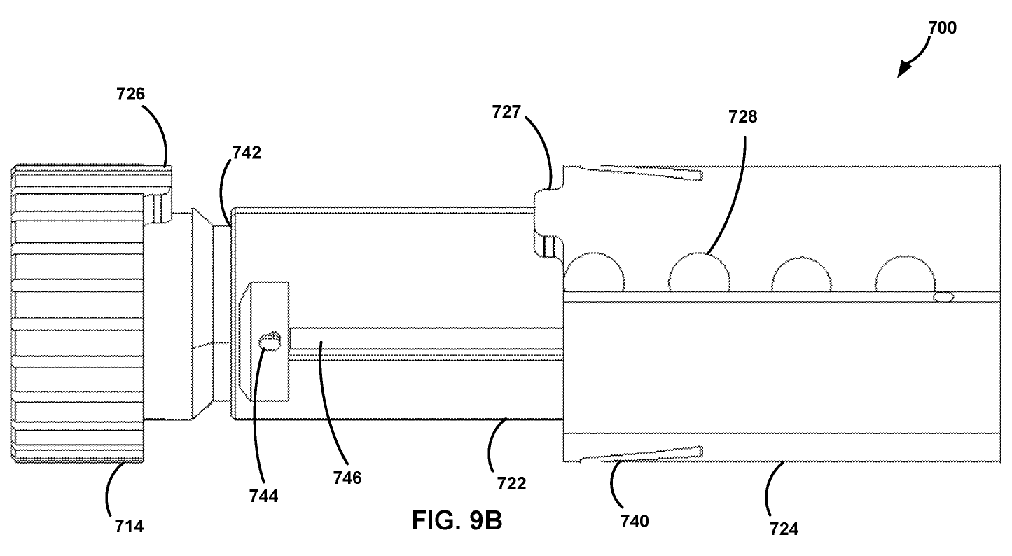

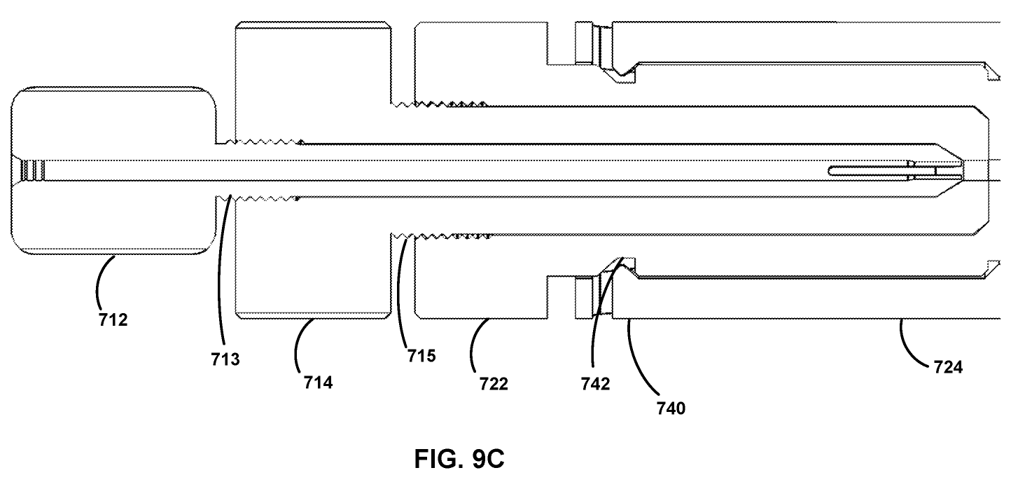

FIG. 9A is a rotated side view of cleaning device 700 of FIG. 7A. FIG. 9B is an exploded side view illustrating the example cleaning device of FIG. 9A. FIG. 9C is a cross-sectional view of the example cleaning device of FIG. 9A. FIGS. 9A, 9B, and FIG. 9C are described together for clarity.

Cleaning device 700 may include lead retention sleeve 712, chuck tightener 714, cleaning actuator 722, and wrapping actuator 724. Cleaning device 700 may utilize a threaded compression mechanism to secure the lead. Lead retention sleeve 712 may include sleeve threaded interface 713 configured to engage chuck tightener 714. Engagement of sleeve threaded interface 713 may facilitate chuck tightener 714 compressing lead retention sleeve 712 onto a distal portion of a lead which may rotationally secure the lead. In other examples, the securing mechanism may include a cam lever, a sliding wedge, or a friction-fit taper. Wrapping actuator 724 may include cleaning grip 728 configured to assist a user in rotating the device. Cleaning grip 728 may be formed as a series of bumps, hemispherical protrusions, ribs, or a soft-touch overmold.

The actuation assembly may include features to limit rotation. Cleaning actuator 722 may include cleaning mechanical stop 726, and wrapping actuator 724 may include wrapping mechanical stop 727. When wrapping actuator 724 is rotated relative to cleaning actuator 722 to wrap the strands, wrapping mechanical stop 727 may contact cleaning mechanical stop 726. This contact provides a hard stop that defines a maximum engagement state. This physical limitation may provide the advantage of wrapping the strands sufficiently to clean the helix but not so tightly that the strands crush or deform the helix coil.

Cleaning actuator 722 may define spring channel 746 extending longitudinally along an outer surface of cleaning actuator 722. Spring channel 746 may be a groove, a slot, or a recess configured to house a spring element or guide a portion of the strand. Spring hook 744 may be positioned within or adjacent to spring channel 746. Spring hook 744 may serve as an anchor point for a spring (not shown) that may bias the strands toward a specific tension level. Snap joint hook 740 on wrapping actuator 724 may interface with snap joint recess 742 on cleaning actuator 722 to rotatably couple the components while avoiding axial separation. Snap joint hook 740 and snap joint recess 742 may be configured to enable rotational movement while restricting axial movement which may enable strands to wrap around patient tissue of a helix.

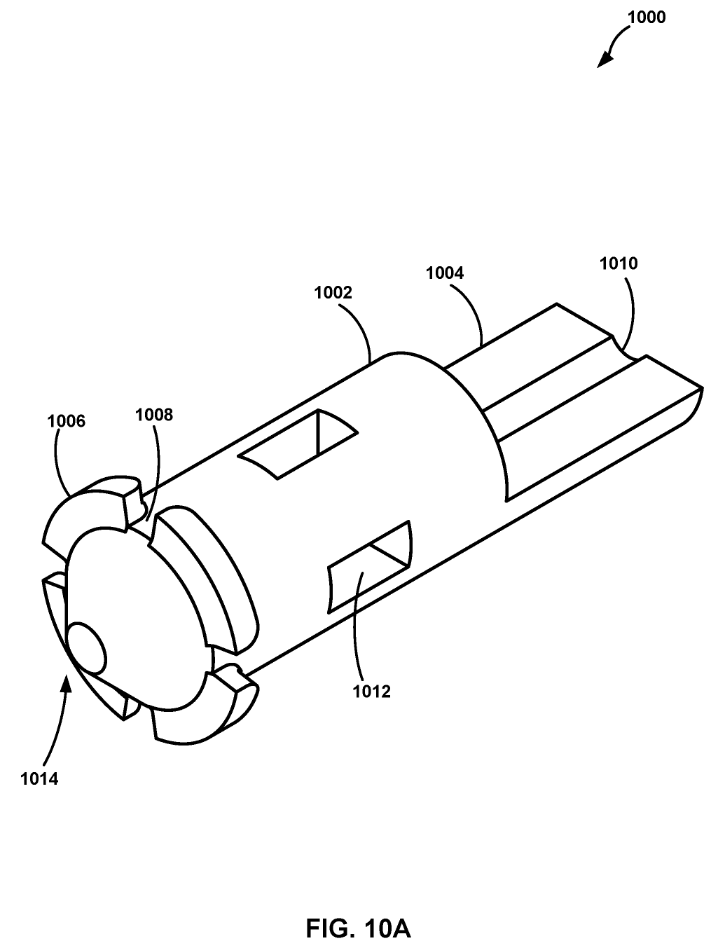

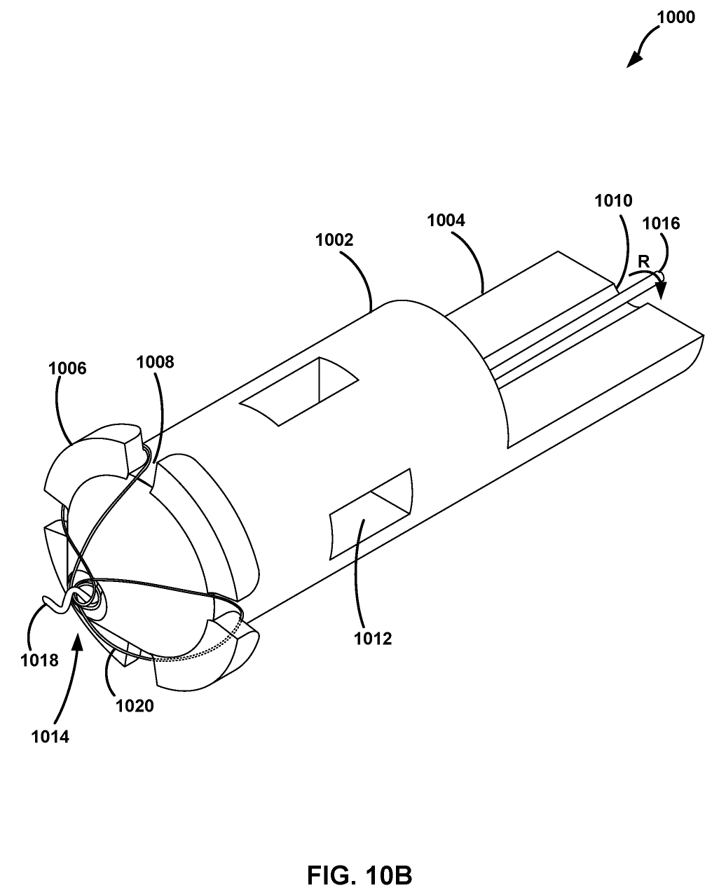

FIG. 10A is a perspective view illustrating an example handheld cleaning tool. FIG. 10B is a perspective view illustrating the example handheld cleaning tool of FIG. 10A with a strand wrapped in an engagement pattern around a helix of a lead. FIGS. 10A and 10B are described together for clarity.

FIG. 10A is a perspective view of cleaning device 1000 in a simplified, unloaded state. FIG. 10B is a perspective view of cleaning device 1000 with one or more strands 1020 wrapped in an engagement pattern around helix 1018 of lead 1016. Cleaning device 1000 may include frame 1002 extending from a proximal end to a distal end. A proximal portion of frame 1002 may define grip 1004 configured to be held by a user. Grip 1004 may include ergonomic features, such as a flattened surface, texturing, or finger grooves, to facilitate stable handling during the cleaning procedure.

Frame 1002 may define a lead slot 1010 extending longitudinally along the device. Lead slot 1010 may be sized to receive lead 1016. In the example shown, lead slot 1010 is an open channel or groove along grip 1004 and transitions to a cylinder for a distal portion of lead slot 1010. The open channel or groove along grip 1004 may facilitate lead 1016 being press-fit or slid laterally into frame 1002. In other examples, lead slot 1010 may be an enclosed lumen where lead 1016 may be threaded through from the distal end. Frame 1002 may optionally further define one or more alignment windows 1012 intersecting lead slot 1010. Alignment window 1012 facilitates a user to visualize lead 1016 to verify insertion depth. For example, the user may align a radiopaque or colored marker or electrode ring or other structure on lead 1016 with alignment window 1012. This visual confirmation may provide the advantage of facilitating helix 1018 being properly positioned within an engagement zone 1014 at the distal end of the device, while keeping sensitive components like drug-eluting collars protected within frame 1002.

The distal end of frame 1002 includes engagement zone 1014, which may support the cleaning mechanism. Frame 1002 may include one or more strand holders 1006 defining one or more strand slots 1008. Strand holders 1006 may be formed as castellated projections, tabs, hooks, or cleats arranged circumferentially around engagement zone 1014. One or more strands1020 (shown in FIG. 10B) may be anchored to frame 1002 and routed through strand slots 1008. Strand holders 1006 and strand slots 1008 together may define an actuation assembly which may enable strand 1020 to transition from a first configuration to a second configuration. In some examples, strands 1020 may be anchored to frame 1002 by tying an end of the one or more strands 1020 to frame 1002. In other examples, one or more ends of strands 1020 may be molded to frame 1002. Strand slots 1008 may function as guides to position one or more strands 1020 in a specific pattern across the face of engagement zone 1014.

FIG. 10B illustrates the device in use. A user may insert lead 1016 into lead slot 1010 and aligns helix 1018 into engagement zone 1014 using alignment window 1012. For example, the user may align a feature of lead 1016 into alignment window 1012 which may indicate that helix 1018 is in an engagement zone 1014. The user may then manually wrap one or more strands 1020 around helix 1018, using strand holders 1006 to secure the wrap pattern. Alternatively, one or more strands 1020 may be pre-strung across engagement zone 1014, and the user may press helix 1018 against one or more strands 1020. The user may apply manual pressure to lead 1016 (e.g., pressing a thumb against the lead body within lead slot 1010) to secure the lead relative to frame 1002. To clean the device, the user may rotate lead 1016 in a direction R (e.g., counter-clockwise) while maintaining the engagement of one or more strands 1020. This rotation unscrews helix 1018 from the web of one or more strands 1020, stripping away tissue. This manual approach may provide the advantage of tactile feedback during the cleaning process which may enable the clinician to adjust the wrapping tension or rotation speed based on the resistance encountered.

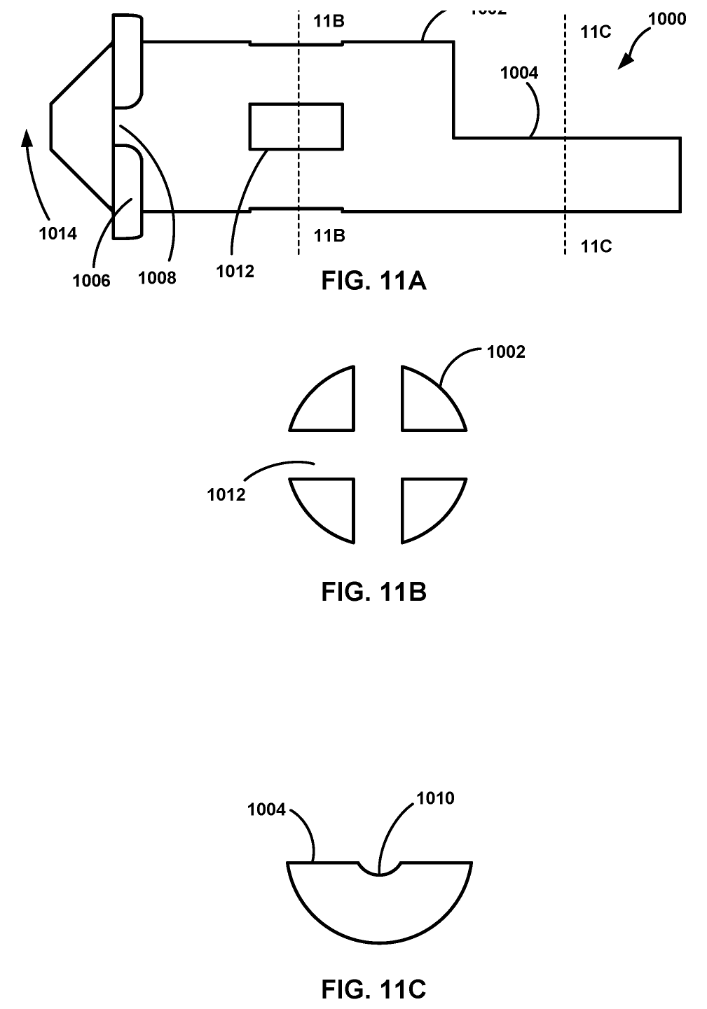

FIG. 11A is a side view of cleaning device 1000 of FIG. 10A. FIG. 11B is a cross-sectional view taken along line 11 B- 11 B of FIG. 11A. FIG. 11C is a cross-sectional view taken along line 11 C- 11 C of FIG. 11A. FIGS. 11A, 11B, and FIG. 11C are described together for clarity.

Cleaning device 1000 may include frame 1002, grip 1004, strand holder 1006, lead slot 1010, alignment window 1012, and engagement zone. The distal end of frame 1002 defines engagement zone 1014. Strand holder 1006 may include a plurality of projections or walls defining strand slot 1008. Strand holder 1006 may extend distally beyond the floor of lead slot 1010 to create a basket-like or cup-like structure for receiving the helix. The strand slots 1008 enable a strand (not shown) to be woven or wrapped transversely across the opening of engagement zone 1014. In other examples, strand holder 1006 may include adhesive patches, molded cleats, or friction slits to secure the strand.

FIG. 11B illustrates the cross-sectional profile of frame 1002 at the location of alignment window 1012. Alignment window 1012 may provide a line of sight into lead slot 1010. Alignment window 1012 is positioned proximally relative to engagement zone 1014. This spacing is calibrated such that when a specific feature of the lead (e.g., an electrode ring) is visible in alignment window 1012, the helix is correctly positioned in engagement zone 1014.

FIG. 11C illustrates the cross-sectional profile of frame 1002 at the location of grip 1004. Frame 1002 defines an elongated body that supports the functional elements of the device. Lead slot 1010 may be defined as a U-shaped or C-shaped channel open to a top side of frame 1002. This open configuration enables the medical lead to be pressed down into lead slot 1010 rather than threaded through a closed lumen. Grip 1004 is located at a proximal end of frame 1002 and may have a profile configured for manual stability. Grip 1004 may have a substantially rectangular, oval, or ergonomic cross-section that resists rotation in a hand of a user. This geometric stability may provide the advantage of facilitating the user to firmly hold the device stationary while rotating the medical lead against the resistance of the engagement strands.

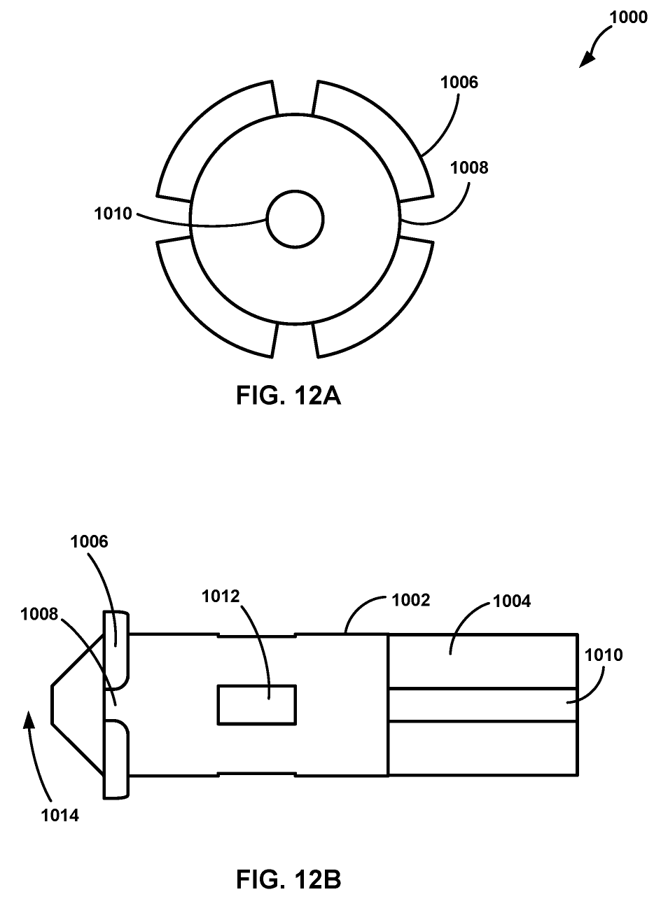

FIG. 12A is a front view of cleaning device 1000 of FIG. 10A. FIG. 12B is a top view of the example cleaning tool of FIG. 10A. FIGS. 12A and 12B are described together for clarity.

Cleaning device 1000 may include frame 1002, grip 1004, strand holder 1006, strand slot 1008, lead slot 1010, alignment window 1012, and engagement zone 1014. Strand holder 1006 may be arranged circumferentially around engagement zone 1014. In this example, strand holder 1006 includes four distinct segments separated by strand slots 1008, though in other examples, there may be two, three, six, or more segments. The radial arrangement of strand slots 1008 may provide the advantage of enabling a user to create various webbing patterns (e.g., an X-pattern, a star pattern, or a parallel wrap) with the strand to optimize the cleaning interface for different helix sizes. Engagement zone 1014 may be centered within strand holders 1006 to receive the distal tip of the lead.

FIG. 12B illustrates the longitudinal profile of frame 1002. Lead slot 1010 extends along the length of frame 1002. Alignment window 1012 intersects lead slot 1010. As depicted, alignment window 1012 may be a rectangular aperture, but in other examples, it may be oval, circular, or graduated with measurement markings. The positioning of alignment window 1012 relative to engagement zone 1014 may facilitate the helix sufficiently protruding into engagement zone 1014 when a feature of a lead is in alignment window 1012.

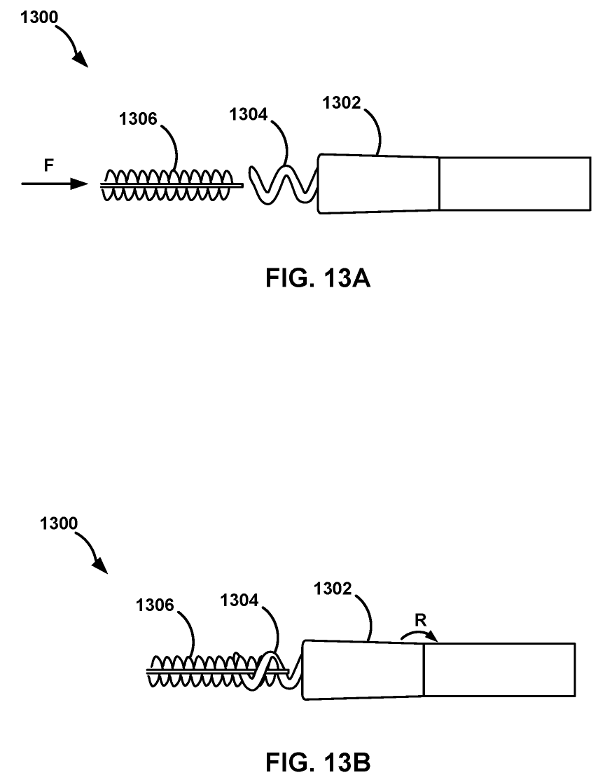

FIG. 13A is a conceptual diagram illustrating an example cleaning tool which includes a brush element in a pre-insertion state. FIG. 13B is a conceptual diagram illustrating the example cleaning tool of FIG. 13A engaging a helix. FIGS. 13A and 13B are described together for clarity.

Linear brush cleaning device 1300 includes a linear brush 1306. Linear brush 1306 may generally define a cylindrical or tapered profile configured to fit within an internal diameter of helix 1304. Linear brush 1306 may include a central spine supporting a plurality of bristles extending radially outward. In other examples, linear brush 1306 may include a twisted-wire brush or a foam cylinder mounted on a shaft. The bristles may be constructed from nylon, polypropylene, or any another stiff biocompatible material capable of mechanically disrupting tissue.

FIG. 13A illustrates a pre-insertion state where linear brush 1306 is aligned with a longitudinal axis of lead 1302. To engage the device, a user may apply a force F to move linear brush 1306 distally toward helix 1304. Alternatively, the user may move lead 1302 toward linear brush 1306. As shown in FIG. 13B, linear brush 1306 may be inserted into the hollow interior space defined by the coils of helix 1304. The bristles of linear brush 1306 may extend radially to contact inner surfaces of helix 1304. This internal engagement may provide the advantage of removing patient tissue that has accumulated inside the coil structure, which may be difficult to access with external wiping methods.

Once linear brush 1306 is inserted, the user may rotate lead 1302 in a direction R (e.g., counter-clockwise) to unscrew helix 1304 from linear brush 1306. Alternatively, the user may pull linear brush 1306 longitudinally to scrub the inner surfaces. As helix 1304 moves relative to linear brush 1306, the bristles may capture and displace the patient tissue. In some examples, linear brush cleaning device 1300 may further include a handle (not shown) or a frame similar to those described in previous examples to stabilize linear brush 1306 during the cleaning process.

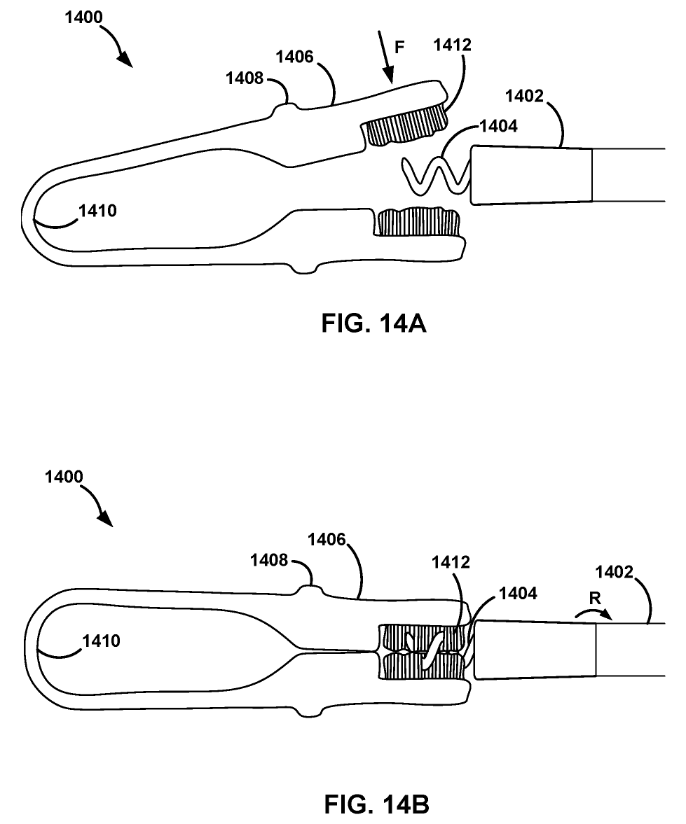

FIG. 14A is a side view illustrating an example tweezer-style cleaning device in a loading configuration. FIG. 14B is a side view illustrating the example tweezer-style cleaning device of FIG. 14A in a cleaning configuration. FIGS. 14A and 14B are described together for clarity.

The tweezer-style cleaning device 1400 may include tweezer 1406 defined by a pair of arms coupled at hinge 1410. Hinge 1410 may be an actuation element, e.g., actuation assembly, which may enable the pair of arms of tweezer 1406 to transition from a first configuration to a second configuration. Hinge 1410 may be a living hinge formed from a flexible polymer. In other examples, hinge 1410 is a mechanical pivot pin connecting two separate arm components. The arms of tweezer 1406 may include one or more finger rests 1408, which may be contoured surfaces, pads, or ridges configured to accept a finger and a thumb of a user.

A distal portion of tweezer 1406 may support an engagement member. In some examples, the engagement member may include a plurality of bristles 1412. Bristles 1412 may be arranged in opposing pads on the inner surfaces of the distal arm ends. Bristles 1412 may be constructed from nylon, stiff cotton, or a polymer material selected to be non-abrasive to the material of helix 1404 while being sufficiently stiff to dislodge patient tissue. In other examples, the engagement member may include a foam block, a sponge material, a textured rubber pad, or a scouring mesh instead of or in addition to the bristles 1412. Bristles 1412 may include any suitable medical-grade brush element designed to capture patient tissue.

FIG. 14A illustrates the device in an open, loading configuration where the opposing pads of bristles 1412 are spaced apart. A user may position lead 1402 such that helix 1404 is located between the opposing pads. As shown in FIG. 14B, to clean helix 1404, the user may apply a compressive force F to finger rests 1408. Force F may pivot the arms of tweezer 1406 about hinge 1410, bringing bristles 1412 together to clamp around helix 1404. Bristles 1412 may interdigitate or compress around the coils of helix 1404. While maintaining force F, the user may rotate lead 1402 in a direction R (e.g., counter-clockwise) to unscrew helix 1404 from stationary bristles 1412. This manual clamping and rotation action may provide the advantage of facilitating the user to tactilely modulate the pressure applied to helix 1404 to facilitate sufficient cleaning force without crushing the helix structure. In some examples, tweezer 1406 may further include a biasing member, such as a spring, coupled to the arms. The biasing member may be configured to apply a consistent, predetermined clamping pressure to helix 1404 when tweezer 1406 is actuated, which may reduce variability associated with manual user force.

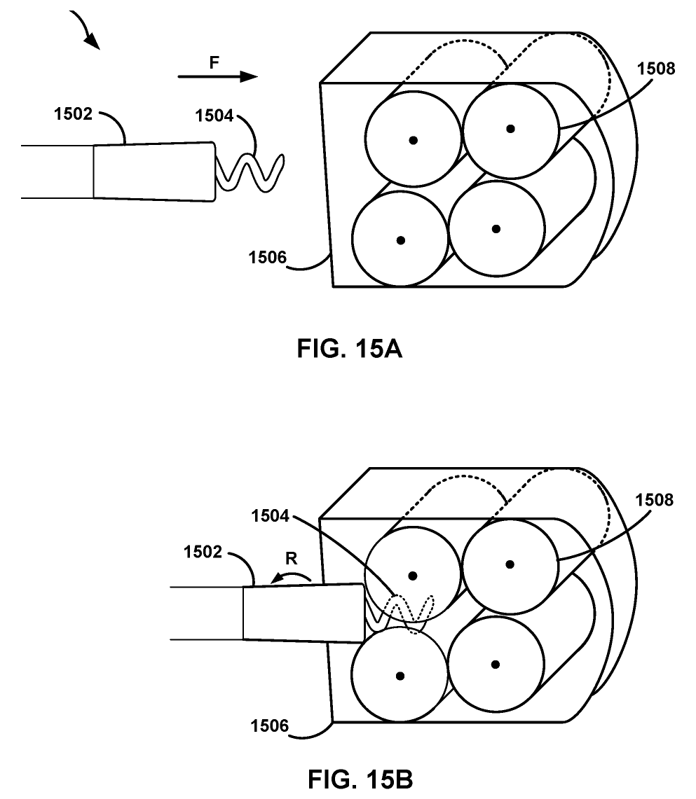

FIG. 15A is a conceptual diagram illustrating an example cleaning device including a foam material in a loading state. FIG. 15B is a conceptual diagram illustrating the example cleaning device of FIG. 15A engaging a helix. FIGS. 15A and 15B are described together for clarity.

Foam cleaning device 1500 may include foam holder 1506 supporting a cleaning element defined by foam 1508. Foam holder 1506 may be a rigid frame, a flexible backing, or a tweezer-like structure. Foam 1508 may include a block, a pad, a sheet, or a cylindrical insert constructed from a compressible foam material. Examples of the material for foam 1508 may include open-cell polyurethane, closed-cell polyethylene, a cellulose sponge, or a melamine foam. Foam 1508 may be configured to be used in a dry state or a wet state. In some examples, foam 1508 may include a fluid, such as distilled water or saline, absorbed into foam 1508 prior to use. This fluid absorption may provide the advantage of softening patient tissue on helix 1504 and lubricating the surface of foam 1508.

To operate foam cleaning device 1500, a user may position helix 1504 against or within foam 1508. As indicated by force F, the user may apply a compressive force to foam holder 1506 or directly to foam 1508. This force may cause foam 1508 to deform and envelop helix 1504, conforming to the external profile and entering the spaces between the coils of helix 1504. While maintaining the compression, the user may rotate lead 1502 in a direction R (e.g., counter-clockwise). This rotation causes helix 1504 to track through foam 1508, effectively unscrewing helix 1504 from the cleaning medium. As helix 1504 exits foam 1508, the porous structure of foam 1508 may capture and retain patient tissue. This method may provide the advantage of cleaning complex geometries of helix 1504 without requiring precise alignment of a rigid cleaning tool.

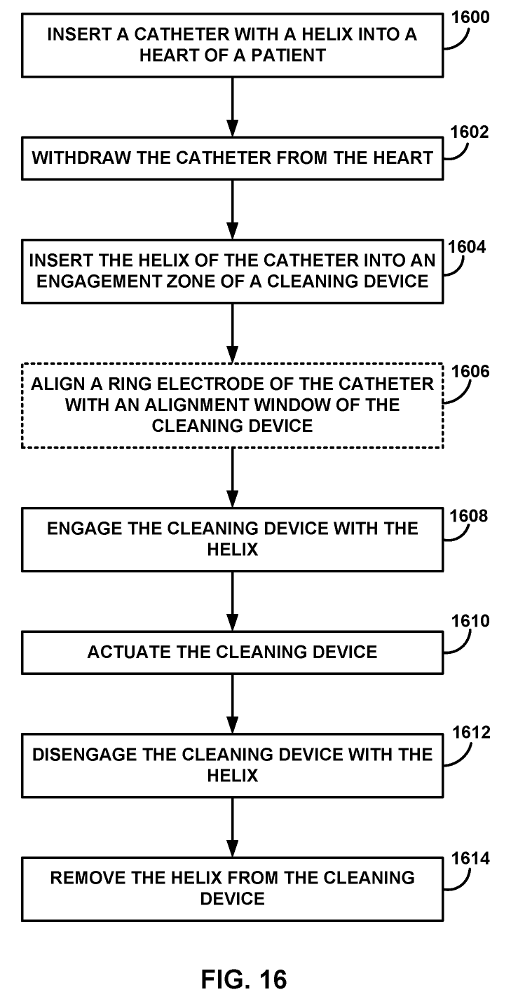

FIG. 16 is a flowchart illustrating an example method for removing patient tissue from a helix of a medical lead. While the description below describes the method with specific reference to the cleaning device 300 illustrated in FIGS. 3A and 3B, the method may be performed using any of the cleaning devices described herein, including, cleaning device 700, cleaning device 1000, the linear brush cleaning device 1300, the tweezer bristle cleaning device 1400, and the foam cleaning device 1500.

The method may begin with inserting a catheter or a medical lead which may include a helix into a heart of a patient (1600). Although the techniques of this disclosure are described with respect to a helix, e.g., a fixation helix, any fixation element may be cleaned by the devices, systems, and techniques described herein. If the initial positioning is determined to be unsatisfactory, or if the helix becomes clogged with patient tissue during navigation, the user withdraws the catheter or medical lead from the heart (1602). To clean the helix, the user inserts the helix 334 of lead 330 into the engagement zone 326 of the cleaning device 300 (1604). Step 1604 may include sliding the distal tip of lead 330 into the lead slot 322 defined by the lead chuck 320. In some examples, the method may include aligning a lead feature 332, such as a ring electrode, with the alignment window 324 of the cleaning device 300 (1606). This alignment step may provide the advantage of facilitating helix 334 being properly aligned with engagement zone 326 while keeping sensitive components, such as a drug-eluting collar, outside of the engagement area to avoid damage.

Once positioned, the user engages the cleaning device 300 with the helix 334 (1608). Engaging the device may include applying a manual compressive force F to first frame portion 302 and second frame portion 304. Force F (e.g., Force F of FIG. 3B) may pivot the frame portions about pivot 306, moving inner top arm 310, outer bottom arm 312, outer top arm 314, and inner bottom arm 316 relative to one another. This movement causes strands 328 anchored in strand slots 318 to tighten and constrict around helix 334 within engagement zone 326. The user may then actuate the cleaning device 300 (1610) to remove the patient tissue. Actuation may involves rotating lead 330 in a direction R (e.g., direction R of FIG. 3B) relative to the cleaning device 300 while maintaining the compressive force F. This relative rotation causes the helix 334 to unscrew from engagement with strands 328, stripping away the immobilized patient tissue. During this rotation, the user may grasp lead 330 at a specific location to reduce torsional energy buildup within the lead body. For example, the user may grasp a lead sleeve head or a rigid portion of the distal assembly located between a defibrillation coil and helix 334. The lead sleeve head may be defined as a segment of lead 330 located between a coil electrode 35 and the helix 334. This segment may include a rigid or semi-rigid spacer configured to transmit torque more effectively than the flexible body of lead 330. Grasping lead 330 at this distal location may reduce the torsional energy stored in the lead body and may avoid lead 330 from winding up, kinking, “coiling,” or twisting that may otherwise occur if the lead were rotated from a more proximal position.

After the tissue is removed, the user may disengage cleaning device 300 from helix 334 (1612). This may include releasing force F on first frame portion 302 and second frame portion 304, enabling inner top arm 310, outer bottom arm 312, outer top arm 314, and inner bottom arm 316 to move relative to one another and loosening strands 328. Finally, the user removes helix 334 from cleaning device 300 (1614) by withdrawing lead 330 from lead slot 322. Lead 330 may then be ready for a subsequent implantation attempt.

The techniques described in this disclosure may be implemented, at least in part, in hardware, software, firmware, or any combination thereof. For example, various aspects of the techniques may be implemented within one or more microprocessors, Digital Signal Processors (DSPs), Application Specific Integrated Circuits (ASICs), Field Programmable Gate Arrays (FPGAs), or any other equivalent integrated or discrete logic circuitry, as well as any combinations of such components, embodied in external devices, such as clinician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as Random Access Memory (RAM), Dynamic Random Access Memory (DRAM), Static Random Access Memory (SRAM), magnetic discs, optical discs, flash memories, or forms of Erasable Programmable Read-Only Memory (EPROM) or Electrically Erasable Programmable Read-Only Memory (EEPROM). The instructions may be executed to support one or more aspects of the functionality described in this disclosure.

In addition, in some respects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an Integrated Circuit (IC) or a set of ICs, and/or discrete electrical circuitry, residing in an IMD and/or external programmer.

The following examples are illustrative of the techniques described herein.

Example 1. A device comprising a frame defining an engagement zone configured to receive a distal portion of a medical lead, the medical lead comprising a helix at a distal end of the medical lead, an engagement element coupled to the frame, and an actuation assembly configured to transition the engagement element from a first configuration to a second configuration, wherein the engagement element in the second configuration is configured to contact the helix such that relative movement of the helix and the engagement element removes patient tissue from the helix.

Example 2. The device of Example 1, wherein the first configuration is a loading configuration, the second configuration is a cleaning configuration, and the actuation assembly is configured to facilitate entry of the distal end of the medical lead into the engagement zone in the loading configuration, and engage the engagement element with the helix of the medical lead in the cleaning configuration.

Example 3. The device of Example 1, wherein the engagement element comprises two or more strands coupled to the frame, wherein the two or more strands intersect at the engagement zone.

Example 4. The device of Example 3, wherein the first configuration is a loading configuration, the second configuration is a cleaning configuration, and the actuation assembly is configured to facilitate entry of the distal end of the medical lead into the engagement zone in the loading configuration, and apply a tension to the two or more strands in the cleaning configuration, wherein the tension of the two or more strands is applied to the helix of the medical lead.

Example 5. The device of Example 4, wherein in the cleaning configuration, the two or more strands constrict around the helix of the medical lead and wherein the two or more strands are configured to immobilize the patient tissue while enabling the helix to rotate.

Example 6. The device of any of Examples 4-5, wherein the two or more strands are arranged in a crisscross pattern, wherein the crisscross pattern is configured to circumscribe the distal end of the medical lead in the second configuration.

Example7. The device of any of Examples 4-6, wherein the frame comprises an inner top arm, an outer top arm, an inner bottom arm, and an outer bottom arm, the two or more strands comprise a first strand set and a second strand set, the first strand set spans from the inner top arm to the outer bottom arm, and the second strand set spans from the outer top arm to the inner bottom arm.

Example 8. The device of any of Examples 4-7, wherein the frame defines a plurality of slots configured to anchor the two or more strands.

Example 9. The device of any of Examples 4-8, wherein the frame is configured to shield a drug-eluting collar of the medical lead from contact with the two or more strands.

Example 10. The device of any of Examples 4-9, wherein the two or more strands are configured to conform to a pitch of a helix of the medical lead.

Example 11. The device of any of Examples 4-10, wherein the two or more strands comprise a biocompatible suture material.

Example 12. The device of any of Examples 4-11, further comprising a mechanical stop configured to limit the tension.

Example 13. The device of any of Examples 1-12, wherein the frame comprises a first frame portion and a second frame portion, wherein the actuation assembly is configured to enable the first frame portion and the second frame portion to pivot relative to one another to transition the engagement element between the first configuration and the second configuration.

Example 14. The device of any of Examples 1-13, wherein the frame defines an alignment window, wherein the alignment window is positioned to align with a feature of the medical lead when the helix of the medical lead is positioned in the engagement zone.

Example 15. The device of any of Examples 1-14, wherein the frame is configured to shield a drug-eluting collar of the medical lead from contact with the engagement element.

Example 16. The device of any of Examples 1-15, wherein the engagement element is configured to conform to a pitch of a helix of the medical lead.

Example 17. The device of Example 1, wherein the engagement element comprises a plurality of bristles.

Example 18. The device of Example 1, wherein the engagement element comprises a foam material.

Example 19. The device of Example 18, wherein the engagement element further comprises distilled water on the foam material.

Example 20. The device of Example 1, wherein the frame is defined by a lead retention chuck comprising a lead retention sleeve and a chuck tightener, wherein rotation of the lead retention sleeve relative to the chuck tightener is configured to secure the distal portion of the medical lead, the actuation assembly is distal and radially outwards of the lead retention chuck, and wherein the actuation assembly comprises a wrapping actuator and a cleaning actuator, wherein the wrapping actuator is rotatably coupled to the cleaning actuator, the wrapping actuator is configured to transition from the first configuration to the second configuration comprising rotating one or more strands relative to the helix of the medical lead, wherein the one or more strands are configured to apply a tension to the helix in the second configuration, and the cleaning actuator is rotatably coupled to the chuck tightener via a threaded interface.

Example 21. The device of Example 20, wherein the first configuration defines an open aperture for receiving the helix, the second configuration defines a cleaning configuration, the one or more strands are configured to immobilize the patient tissue in the cleaning configuration, the threaded interface is configured to convert a rotational motion of the cleaning actuator into a longitudinal motion of the actuation assembly relative to the lead retention chuck, and the longitudinal motion removes the patient tissue from the helix.

Example 22. The device of Example 21, wherein the one or more strands comprise two or more strands and the two or more strands are arranged in a crisscross pattern in the cleaning configuration, wherein the crisscross pattern is configured to intersect an engagement zone defined by the actuation assembly.

Example 23. The device of any of Examples 20-22, wherein the one or more strands comprises two strands and the tension is radially balanced.

Example 24. The device of any of Examples 20-23, wherein the threaded interface defines a thread pitch based on a helix pitch of the helix.

Example 25. The device of any of Examples 20-24, wherein the wrapping actuator comprises a mechanical stop configured to define a degree of rotation of the wrapping actuator relative to the cleaning actuator.

Example26. The device of any of Examples 20-25, wherein the actuation assembly further comprises a spring element coupled to the one or more strands, wherein the spring element is configured to modulate the tension.

Example27. The device of any of Examples 20-26, wherein the lead retention sleeve comprises a collet configured to radially compress against the distal portion of the medical lead.

Example 28. The device of any of Examples 20-27, wherein the actuation assembly is configured to shield a drug-eluting component located on a distal portion of the medical lead from contact with the one or more strands.

Example 29. The device of any of Examples 20-28, wherein the lead retention chuck includes a grip surface configured for rotation by a user.

Example 30. The device of any of Examples 20-29, wherein the one or more strands comprise a biocompatible suture material.

Example 31. A method comprising inserting a helix of a medical lead into an engagement zone of a frame of a cleaning device, wherein an engagement element of the cleaning device is in a loading configuration, transitioning the cleaning device from the loading configuration to a cleaning configuration, wherein the engagement element is configured to immobilize patient tissue in the helix in the cleaning configuration, and rotating the medical lead relative to the engagement element while maintaining the cleaning configuration to separate the patient tissue from the helix.

Example 32. The method of Example 31, wherein inserting the helix comprises inserting the medical lead into a lead slot of the frame until a feature of the medical lead is visible through an alignment window of the cleaning device and the helix is in the engagement zone.

Example 33. The method of any of Examples 31-32, wherein rotating the medical lead comprises rotating a distal portion of the medical lead while holding the cleaning device stationary.

Example 34. The method of any of Examples 31-33, wherein the engagement element comprises one or more strands.

Example 35. The method of Example 34, wherein transitioning the cleaning device comprises actuating a frame of the cleaning device, wherein actuating the frame comprises compressing a tweezer mechanism defining pivotable arms of the frame to constrict the one or more strands.

Example 36. The method of any of Examples 34-35, further comprising limiting a tension applied to the one or more strands using a mechanical stop integrated into the frame to avoid damage to the helix.

Example 37. The method of any of Examples 34-36, wherein the one or more strands comprise a first strand set and a second strand set, and transitioning to the cleaning configuration is configured to apply a balanced constriction force from opposing sides of the helix.

Example 38. The method of any of Examples 34-37, wherein the one or more strands are arranged in a crisscross pattern, and wherein transitioning to the cleaning configuration causes the crisscross pattern to encircle the helix of the medical lead.

Example 39. The method of Example 31, wherein the engagement element comprises one or more strands, wherein the cleaning device comprises a lead retention chuck and a cleaning assembly, and the method further comprises securing a distal portion of the medical lead within a lead retention chuck by rotating a lead retention sleeve of the lead retention chuck relative to a chuck tightener to radially compress a distal portion of the medical lead.

Example 40. The method of Example 39, wherein transitioning the cleaning device comprises rotating a wrapping actuator of the cleaning assembly relative to a cleaning actuator until a mechanical stop is engaged to wrap the one or more strands around the helix at a predetermined degree of constriction.

Example 41. The method of any of Examples 39-40, wherein rotating the medical lead relative to the one or more strands is defined by rotating the lead retention chuck relative to the cleaning assembly via a threaded interface, the threaded interface defines a thread pitch corresponding to a helix pitch of the helix, and rotating the lead retention chuck causes a longitudinal withdrawal of the helix from the engagement zone.

Example 42. The method of any of Examples 39-41, wherein the cleaning assembly further comprises a spring element configured to moderate a tension of the one or more strands against the helix.

Example 43. The method of any of Examples 39-42, wherein the one or more strands are arranged in a crisscross pattern to intersect the engagement zone.

Example 44. The method of Example 31, wherein the engagement element comprises a plurality of bristles.

Example 45. The method of Example 31, wherein the engagement element comprises a foam material.

Example 46. The method of Example 45, wherein the engagement element further comprises distilled water on the foam material.

Example 47. A system comprising a medical lead comprising a helix at a distal end of the medical lead, and a cleaning device comprising a frame defining an engagement zone configured to receive the distal end of the medical lead, an engagement element coupled to the frame, and an actuation assembly configured to transition the engagement element from a first configuration to a second configuration, wherein the engagement element in the second configuration contacts the helix such that relative movement of the helix and the engagement element removes patient tissue from the helix.

Example 48. The system of Example 47, wherein the first configuration is a loading configuration, the second configuration is a cleaning configuration, and the actuation assembly is configured to facilitate entry of the distal end of the medical lead into the engagement zone in the loading configuration, and engage the engagement element with the helix of the medical lead in the cleaning configuration.

Example 49. The system of any of Examples 47-48, wherein the engagement element comprises two or more strands coupled to the frame, wherein the two or more strands intersect at the engagement zone.

Example 50. The system of Example 49, wherein the first configuration is a loading configuration, the second configuration is a cleaning configuration, and the actuation assembly is configured to facilitate entry of the distal end of the medical lead into the engagement zone in the loading configuration, and apply a tension to the two or more strands in the cleaning configuration, wherein the tension of the two or more strands is applied to the helix of the medical lead.

Example 51. The system of any of Examples 47-50, wherein the frame comprises a first frame portion and a second frame portion, wherein the actuation assembly is configured to enable the first frame portion and the second frame portion to pivot relative to one another to transition the engagement element between the first configuration and the second configuration, and wherein the actuation assembly further comprises a mechanical stop configured to limit a tension applied by the engagement element.

Example 52. The system of any of Examples 47-51, wherein the frame defines an alignment window, wherein the alignment window is positioned to align with a feature of the medical lead when the helix of the medical lead is positioned in the engagement zone.

Example 53. The system of any of Examples 47-52, wherein the medical lead further comprises a drug-eluting collar, and wherein the frame is configured to shield the drug-eluting collar from contact with the engagement element.

Example 54. The system of any of Examples 47-53, wherein the engagement element is configured to conform to a pitch of the helix of the medical lead.

Example 55. The system of Example 47, wherein the engagement element comprises a plurality of bristles.

Example 56. The system of Example 47, wherein the engagement element comprises a foam material.

Example 57. The system of Example 56, wherein the engagement element further comprises distilled water on the foam material.

Various examples have been described. These and other examples are within the scope of the following claims.

What is claimed is:

1. A device comprising:
a frame defining an engagement zone configured to receive a distal portion of a medical lead, the medical lead comprising a helix at a distal end of the medical lead;
an engagement element coupled to the frame; and
an actuation assembly configured to transition the engagement element from a first configuration to a second configuration, wherein the engagement element in the second configuration is configured to contact the helix such that relative movement of the helix and the engagement element removes patient tissue from the helix.

2. The device of claim 1, wherein:
the first configuration is a loading configuration;
the second configuration is a cleaning configuration; and
the actuation assembly is configured to:
facilitate entry of the distal end of the medical lead into the engagement zone in the loading configuration; and
engage the engagement element with the helix of the medical lead in the cleaning configuration.

3. The device of claim 1, wherein the engagement element comprises two or more strands coupled to the frame, wherein the two or more strands intersect at the engagement zone.

4. The device of claim 3, wherein:
the first configuration is a loading configuration;
the second configuration is a cleaning configuration; and
the actuation assembly is configured to:
facilitate entry of the distal end of the medical lead into the engagement zone in the loading configuration; and
apply a tension to the two or more strands in the cleaning configuration, wherein the tension of the two or more strands is applied to the helix of the medical lead.

5. The device of claim 4, wherein in the cleaning configuration, the two or more strands constrict around the helix of the medical lead and wherein the two or more strands are configured to immobilize the patient tissue while enabling the helix to rotate.

6. The device of claim 4, wherein the two or more strands are arranged in a crisscross pattern, wherein the crisscross pattern is configured to circumscribe the distal end of the medical lead in the second configuration.

7. The device of claim 4, wherein:
the frame comprises an inner top arm, an outer top arm, an inner bottom arm, and an outer bottom arm;
the two or more strands comprise a first strand set and a second strand set;
the first strand set spans from the inner top arm to the outer bottom arm; and
the second strand set spans from the outer top arm to the inner bottom arm.

8. The device of claim 4, wherein the frame defines a plurality of slots configured to anchor the two or more strands.

9. The device of claim 4, wherein the frame is configured to shield a drug-eluting collar of the medical lead from contact with the two or more strands.

10. The device of claim 4, wherein the two or more strands are configured to conform to a pitch of a helix of the medical lead.

11. The device of claim 4, wherein the two or more strands comprise a biocompatible suture material.

12. The device of claim 4, further comprising a mechanical stop configured to limit the tension.

13. The device of claim 1, wherein the frame comprises a first frame portion and a second frame portion, wherein the actuation assembly is configured to enable the first frame portion and the second frame portion to pivot relative to one another to transition the engagement element between the first configuration and the second configuration.

14. The device of claim 1, wherein the frame defines an alignment window, wherein the alignment window is positioned to align with a feature of the medical lead when the helix of the medical lead is positioned in the engagement zone.

15. The device of claim 1, wherein the frame is configured to shield a drug-eluting collar of the medical lead from contact with the engagement element.

16. The device of claim 1, wherein the engagement element is configured to conform to a pitch of a helix of the medical lead.

17. The device of claim 1, wherein the engagement element comprises a plurality of bristles.

18. The device of claim 1, wherein the engagement element comprises a foam material.

19. The device of claim 18, wherein the engagement element further comprises distilled water on the foam material.

20. The device of claim 1, wherein:
the frame is defined by a lead retention chuck comprising a lead retention sleeve and a chuck tightener, wherein rotation of the lead retention sleeve relative to the chuck tightener is configured to secure the distal portion of the medical lead,
the actuation assembly is distal and radially outwards of the lead retention chuck, and wherein the actuation assembly comprises a wrapping actuator and a cleaning actuator, wherein:
the wrapping actuator is rotatably coupled to the cleaning actuator;
the wrapping actuator is configured to transition from the first configuration to the second configuration comprising rotating one or more strands relative to the helix of the medical lead, wherein the one or more strands are configured to apply a tension to the helix in the second configuration; and
the cleaning actuator is rotatably coupled to the chuck tightener via a threaded interface.

21. The device of claim 20, wherein:
the first configuration defines an open aperture for receiving the helix;
the second configuration defines a cleaning configuration;
the one or more strands are configured to immobilize the patient tissue in the cleaning configuration;
the threaded interface is configured to convert a rotational motion of the cleaning actuator into a longitudinal motion of the actuation assembly relative to the lead retention chuck; and
the longitudinal motion removes the patient tissue from the helix.

22. The device of claim 21, wherein the one or more strands comprise two or more strands and the two or more strands are arranged in a crisscross pattern in the cleaning configuration, wherein the crisscross pattern is configured to intersect an engagement zone defined by the actuation assembly.

23. The device of claim 20, wherein the one or more strands comprises two strands and the tension is radially balanced.

24. The device of claim 20, wherein the threaded interface defines a thread pitch based on a helix pitch of the helix.

25. The device of claim 20, wherein the wrapping actuator comprises a mechanical stop configured to define a degree of rotation of the wrapping actuator relative to the cleaning actuator.

26. The device of claim 20, wherein the actuation assembly further comprises a spring element coupled to the one or more strands, wherein the spring element is configured to modulate the tension.

27. The device of claim 20, wherein the lead retention sleeve comprises a collet configured to radially compress against the distal portion of the medical lead.

28. The device of claim 20, wherein the actuation assembly is configured to shield a drug-eluting component located on a distal portion of the medical lead from contact with the one or more strands.

29. The device of claim 20, wherein the lead retention chuck includes a grip surface configured for rotation by a user.

30. The device of claim 20, wherein the one or more strands comprise a biocompatible suture material.

31. A method comprising:
inserting a helix of a medical lead into an engagement zone of a frame of a cleaning device, wherein an engagement element of the cleaning device is in a loading configuration;
transitioning the cleaning device from the loading configuration to a cleaning configuration, wherein the engagement element is configured to immobilize patient tissue in the helix in the cleaning configuration; and
rotating the medical lead relative to the engagement element while maintaining the cleaning configuration to separate the patient tissue from the helix.

32. A system comprising:
a medical lead comprising a helix at a distal end of the medical lead; and
a cleaning device comprising:
a frame defining an engagement zone configured to receive the distal end of the medical lead;
an engagement element coupled to the frame; and
an actuation assembly configured to transition the engagement element from a first configuration to a second configuration, wherein the engagement element in the second configuration contacts the helix such that relative movement of the helix and the engagement element removes patient tissue from the helix.
